SEQUENCE LISTING (350 sequences)

SEQ ID NO: 1 - DNA, 64 bp, Canis familiaris
gctaggcgtgagatcagaggaagcttctggaagaggstgcagttgagctgggccatggacacaa

SEQ ID NO: 2 - DNA, 100 bp, Canis familiaris
tcaaacttagtattgataaattgaactcctgatcttctgctcaacctccarcactgctctgcgctcaattttctgggcaccagccctctcccaaaaggct

SEQ ID NO: 3 - DNA, 103 bp, Canis familiaris
cctttgggtatatttccagaagtagaattactggatcatgtagcatttgtattttyagttttttgaggatttttcatactgttttccatagtggctgcaccag

SEQ ID NO: 4 - DNA, 106 bp, Canis familiaris
tgatttgccacttctggatgtttcatataaatggaatcatgtagccttttgtatctgkcttctttcacttaccctagtgtttctaaggttcatccatattgtagcg

SEQ ID NO: 5 - DNA, 111 bp, Canis familiaris
aaccttggatattgtgtgttaatttctgtattgaaaagtgagggttcacttcatttgtactaccccttccamattttttatagtgaatttattttcagatcttgtattacc

SEQ ID NO: 6 - DNA, 109 bp, Canis familiaris
atatgagaaaaagcaatcccacactacagaggctttttgcaagcatcacagtggrgctgggagaggtggcttcattcagcgcaggagagaggactcggctggcagtgtc

SEQ ID NO: 7 - DNA, 98 bp, Canis familiaris
agctaaaccactaagccaccagggctgcccccaagtcatattttctaaaacatakatatatatgagaaaaagcaatcccacactacagaggctttttg

SEQ ID NO: 8 - DNA, 72 bp, Canis familiaris
tcaatcccagcccctgtacacacttttatggacrtaggagaagggacttcccaaagtcacccagctagaagg

SEQ ID NO: 9 - DNA, 91 bp, Canis familiaris
gggacttcccaaagtcacccagctagaaggtaaggcacagrcccagattttaaatccaggtctaattgcctccgggcgtcctactcttaac

SEQ ID NO: 10 - DNA, 102 bp, Canis familiaris
gggtatatcaatattttagggtcttctcccaaagaacctcttgattttcagmgcttatgggcttgaacatgggttaaaccagtggttctcaaagtgtggtct

SEQ ID NO: 11 - DNA, 81 bp, Canis familiaris
aaacaaggagagaaactaaacctggccaccagatcattgccrtaatttgaaatcacctctaattgtctcccaccaccacca

SEQ ID NO: 12 - DNA, 58 bp, Canis familiaris
tttccctacagccaggcacgactttttaccctacyattgtacacaaaacagacatatc

SEQ ID NO: 13 - DNA, 60 bp, Canis familiaris
cactcgctagccacgctttttaggccaaccccgcrtcgcctctcccaaggcgactgggtg

SEQ ID NO: 14 - DNA, 71 bp, Canis familiaris
acagacgccatagtcttcctataaactcagtyctttaagacattatccttaaactctaaaagatcatgctg

SEQ ID NO: 15 - DNA, 69 bp, Canis familiaris
gtcacagtttactgagcacttattttgagccagccrgtgctagttctgtacatgtcagccatagggtat

SEQ ID NO: 16 - DNA, 81 bp, Canis familiaris
gctcttcctagttactgtcttcactggggaggtargaaaagctcctrtagaaggagaaggtcaaggtacatcaagggaccc

SEQ ID NO: 17 - DNA, 81 bp, Canis familiaris; identical to SEQ ID NO: 16

SEQ ID NO: 18 - DNA, 66 bp, Canis familiaris
ccgccctctcctttccttattagaggtaragcaactttcctcactgcacctgcctaccgcccctgc

SEQ ID NO: 19 - DNA, 81 bp, Canis familiaris
  misc_feature (38)..(38): n is a, c, g, or t
acttggggaaactgaggctcttcccagttcagcaaggnaaaagccttgggtrttcaatccaggttggggaggggatccaat

SEQ ID NO: 20 - DNA, 64 bp, Canis familiaris
acaagctggacaacatactgctgacygggtccctgctggaggactttaaggtgagagcccggct

SEQ ID NO: 21 - DNA, 32 bp, Canis familiaris
taaagggcttttartcagaccagtttcaattc

SEQ ID NO: 22 - DNA, 30 bp, Canis familiaris
gatgagagaggaraatcaggttgggctgtt

SEQ ID NO: 23 - DNA, 30 bp, Canis familiaris
cccacgtgtagcctcrtccccacccaagtg

SEQ ID NO: 24 - DNA, 29 bp, Canis familiaris
agccaggagggyccagcagcccccagccc

SEQ ID NO: 25 - DNA, 28 bp, Canis familiaris
ggtcaaagcccrgggcgagctgaggccc

SEQ ID NO: 26 - DNA, 26 bp, Canis familiaris
aaagtagtgggaycttttccaggaag

SEQ ID NO: 27 - DNA, 67 bp, Canis familiaris
gaaaactaaagtctgagctgcataagctgtttctcctayaggggtgacttgctctgatgctaaacct

SEQ ID NO: 28 - DNA, 67 bp, Canis familiaris
gcttagaaagagaattaagggctcagggctggrcctcaagcttagaactttaaacgacacttagaaa

SEQ ID NO: 29 - DNA, 53 bp, Canis familiaris
ttttgcctgctaacatttcagctggrtttgaaggcttatataaggttgggggg

SEQ ID NO: 30 - DNA, 72 bp, Canis familiaris
agaagctccctgaggagctgtcgtattarttaactgctggaggagaagaaggaggattggataagataatgg

SEQ ID NO: 31 - DNA, 52 bp, Canis familiaris
gcattaggcccgtattccacaragtgtcctctactgtgctgagctatatgga

SEQ ID NO: 32 - DNA, 51 bp, Canis familiaris
tatggacagtgggaaatcataaagtgygggaataggcaatcaccatattcc

SEQ ID NO: 33 - DNA, 73 bp, Canis familiaris
gcattaactgcattttgtccagtcatctttyaatctaagtgcatatcccatatcactggcatatcacaggttc

SEQ ID NO: 34 - DNA, 81 bp, Canis familiaris
gcttgaaaagttccctttagaaagaaaaacatgtytctcctcatatggaaggtttgaatctcttggatcattttggctgac

SEQ ID NO: 35 - DNA, 68 bp, Canis familiaris
ggatcattttggctgactttttttggaccktttccaactctattttgtctttgttaaggcttttaaga

SEQ ID NO: 36 - DNA, 55 bp, Canis familiaris
aaattatcaatgtgctctatggmtgaggactcaacaatttacaaaggcaaaggat

SEQ ID NO: 37 - DNA, 58 bp, Canis familiaris
atgagtaaagttgatgagatmtgtaagaggtatgttgracaaaaagaggaagggggca

SEQ ID NO: 38 - DNA, 58 bp, Canis familiaris; identical to SEQ ID NO: 37

SEQ ID NO: 39 - DNA, 51 bp, Canis familiaris
aagaaatgctggaagccaggctaaaaagagargcattaggcccgtattcca

SEQ ID NO: 40 - DNA, 44 bp, Canis familiaris
agtacatgaaaactcctcmgtattaagcgaggtggtccccaatg

SEQ ID NO: 41 - DNA, 61 bp, Canis familiaris
agccagaggcaaattcattkatttcccgtgatttgggtattttctctcaacaaaatgctaa

SEQ ID NO: 42 - DNA, 63 bp, Canis familiaris
tatggactaaagctgtcatgggtcaaggrctcagaccagcagcttagcagctttggagatgtg

SEQ ID NO: 43 - DNA, 71 bp, Canis familiaris
gaggttatcttttcgacgtaacagctaaacccayggcttcctttctcgtaaaaccaaaacaaaaaggcttt

SEQ ID NO: 44 - DNA, 55 bp, Canis familiaris
taaagatagggaaactgaatcatrggagagttaggatgcttcctcagaatcacat

SEQ ID NO: 45 - DNA, 49 bp, Canis familiaris
ttccttttttacttacttctgaccacaaamaaattatcaatgtgctcta

SEQ ID NO: 46 - DNA, 61 bp, Canis familiaris
cgccacttgaatgtgtcaggtgatatgacytgtgtcctgattaacacatagcatttcttct

SEQ ID NO: 47 - DNA, 54 bp, Canis familiaris
ataatttcataatgattcatgcwgtgtcaaactttttctggggtaaatgaacta

SEQ ID NO: 48 - DNA, 85 bp, Canis familiaris
aaggagggaagggacaggtaagagaaaaaaaaagcgggggggkggggggcctgcagtccagtcttcatggaatcctgacttaact

SEQ ID NO: 49 - DNA, 62 bp, Canis familiaris
aaaagctggaaagttattttaaaacmgagagagaggtagctcatcctaaaatagctgtaatg

SEQ ID NO: 50 - DNA, 64 bp, Canis familiaris
agccagccgacaccagagcaccctacytgaggacgactgcacccacttcccagccagcctgccc

SEQ ID NO: 51 - DNA, 49 bp, Canis familiaris
ccccaacgctyttgcctttrgttacctgggttgccaagccctgtcggag

SEQ ID NO: 52 - DNA, 56 bp, Canis familiaris
agctgtcccccaagtgccagggacacrggagctgggagccgtggcattaacacttt

SEQ ID NO: 53 - DNA, 52 bp, Canis familiaris
ccgcaccctcttcccagaacaggcggcctcsgccctctgcggggctgagccc

SEQ ID NO: 54 - DNA, 44 bp, Canis familiaris
attgtacacaaaacagacatatcmgatatttcctttatctcttc

SEQ ID NO: 55 - DNA, 69 bp, Canis familiaris
cttattcttcttatgatttagtcagyggyttctaaccaygtgtcagagaacatggatgctctctgagat

SEQ ID NO: 56 - DNA, 54 bp, Canis familiaris
catggatgctctctgagatggatggagatgttycaggatgagatgaaatgataa

SEQ ID NO: 57 - DNA, 56 bp, Canis familiaris
taaatatctctacctaattcagawgtagggtacagttttcacattctaaatatttg

SEQ ID NO: 58 - DNA, 54 bp, Canis familiaris
tttaacaaggcttccaggttactttgatgtgyactcaagcttgagaatcactgg

SEQ ID NO: 59 - DNA, 61 bp, Canis familiaris
tctcgctcagagccttttacatagtcartaccaagtatataattgctaaatgttgatccca

SEQ ID NO: 60 - DNA, 62 bp, Canis familiaris
tctccactccctgtgctctccagtttatrttgtagagttggactggcaccctgatgcccccg

SEQ ID NO: 61 - DNA, 58 bp, Canis familiaris
tttctgaaatgtgaggcaaagaatyattctggacgtttcacatgctggtggctgacgg

SEQ ID NO: 62 - DNA, 61 bp, Canis familiaris
aggtcatcttgtgaaggacagaatccaygtgagtgtatgaggaaggccctgcaaccatatt

SEQ ID NO: 63 - DNA, 63 bp, Canis familiaris
gggcagcactctccagttagctcccccacccmcctccatgggaggtggcaagtgtctgcaaag

SEQ ID NO: 64 - DNA, 63 bp, Canis familiaris
ccttaagaatcaggtgacaggctcagcaaggggatsaatgtccccaattcttccatttggcac

SEQ ID NO: 65 - DNA, 77 bp, Canis familiaris
aagaaaacctagggcaagcgtgattcagagcctcagagscttgtctgtgtttggagattccttctcaggcacctctg

SEQ ID NO: 66 - DNA, 61 bp, Canis familiaris
acatgacacagagatccaagtcttcaccagggcccctgcrcagagagcagggctgacgctg

SEQ ID NO: 67 - DNA, 64 bp, Canis familiaris
acgtcttaggttttcacaaatatgaattaactgraatgctaaatcctagcccgctaatctggta

SEQ ID NO: 68 - DNA, 54 bp, Canis familiaris
tagcccgctaatctggtaattaaagtwtttttttaatcatagccttagcttctc

SEQ ID NO: 69 - DNA, 62 bp, Canis familiaris
cccgggacccctggcaggagattccaaggatgaygccacttcaaatagtctaccactcacct

SEQ ID NO: 70 - DNA, 83 bp, Canis familiaris
gcagtcgcaggatgagtggctgaagcacacaacaattcacctcatcctgcrgagtctggaggatttcctgcagttcagtctga

SEQ ID NO: 71 - DNA, 75 bp, Canis familiaris
gtaaagatgcaatcaaaagcctttgaaatgacaaccacttatrtaagacctagcaatgtgcacttccaaacatta

SEQ ID NO: 72 - DNA, 24 bp, Canis familiaris
gctcctatcccrgtaaccaccccc

SEQ ID NO: 73 - DNA, 24 bp, Canis familiaris
cccccaaacamcctaaaatccatc

SEQ ID NO: 74 - DNA, 31 bp, Canis familiaris
cctcttgaccaggggcyattccatcgggtcc

SEQ ID NO: 75 - DNA, 28 bp, Canis familiaris
ggggacgccctcrtggtcaggcctggcc

SEQ ID NO: 76 - DNA, 31 bp, Canis familiaris
ctgaggtcccttccygggccaccccctcccc

SEQ ID NO: 77 - DNA, 30 bp, Canis familiaris
gtggtcaggccacrccggcgccgagcccca

SEQ ID NO: 78 - DNA, 28 bp, Canis familiaris
  misc_feature (5)..(5): n is a, c, g, or t
ggcanggggtggrgtgggcggggcgcgc

SEQ ID NO: 79 - DNA, 31 bp, Canis familiaris
agctcccttcacgcrgggagtctcagaatgt

SEQ ID NO: 80 - DNA, 60 bp, Canis familiaris
aggaaaccttcaacatttatctgccaagagtctgacgtkgtaccacctgaactgggccag

SEQ ID NO: 81 - DNA, 121 bp, Canis familiaris
ctaggagaggaggcagatacatatgcagataacacaagggagtgmaaagaagaatggggaaaatgctgagtgtgggctaagtcattcattaagcttctcaagaagcacaaagcagtggtga

SEQ ID NO: 82 - DNA, 80 bp, Canis familiaris
tgtgttaccaaagctaatgtggtcattaaaacaastgcagagatgtaacaaacagaattacattctcattatcttgtttg

SEQ ID NO: 83 - DNA, 68 bp, Canis familiaris
aaagcagttacatactactcataagctatgttyctccagataataactatgctcctttgtaagttact

SEQ ID NO: 84 - DNA, 59 bp, Canis familiaris
gccttgactctggagtctataacttgtgaygtgttgacagtccacgtgtactatgtaca

SEQ ID NO: 85 - DNA, 64 bp, Canis familiaris
ttgacagtccacgtgtactatgtacatggargagtccaatcctttactcatagtcacttgctga

SEQ ID NO: 86 - DNA, 31 bp, Canis familiaris
aaatgtacaaaaagyaaaataagacaaacac

SEQ ID NO: 87 - DNA, 29 bp, Canis familiaris
ggatacatttagcyaatcagttatgacta

SEQ ID NO: 88 - DNA, 23 bp, Canis familiaris
caaaagaaacrgagtaatagggg

SEQ ID NO: 89 - DNA, 31 bp, Canis familiaris
atgagaatgtataawgggaggtttgctctat

SEQ ID NO: 90 - DNA, 32 bp, Canis familiaris
aatctgaagaactaygaagtgttaactaggta

SEQ ID NO: 91 - DNA, 30 bp, Canis familiaris
tttttttcagctrtttaaaactgtgaaata

SEQ ID NO: 92 - DNA, 21 bp, Canis familiaris
ccccagccctsgggagagata

SEQ ID NO: 93 - DNA, 21 bp, Canis familiaris
atagtgtttrgaatcataatt

SEQ ID NO: 94 - DNA, 28 bp, Canis familiaris
gttttggggtayccagcttgctcaggca

SEQ ID NO: 95 - DNA, 29 bp, Canis familiaris
gcctcttttggctwcataactctcctgca

SEQ ID NO: 96 - DNA, 24 bp, Canis familiaris
ccgaggggggcragtaggaagtat

SEQ ID NO: 97 - DNA, 59 bp, Canis familiaris
ttggagccttcgctctgtagaaaaatccmgaaaaaaaaaattggtttcaagaccttttc

SEQ ID NO: 98 - DNA, 27 bp, Canis familiaris
aaacctcttttctcwgaaatgctgtct

SEQ ID NO: 99 - DNA, 26 bp, Canis familiaris
ccagggctctacmgtctccccactgg

SEQ ID NO: 100 - DNA, 72 bp, Canis familiaris
gggctccagaaggtgcttctgcctcagcctcttctccttcctcctcrtcgcaggggccaccacactcttctg

SEQ ID NO: 101 - DNA, 27 bp, Canis familiaris
cagaccttagagrtggtatgagaggga

SEQ ID NO: 102 - DNA, 23 bp, Canis familiaris
ggagaccccagwggggaccgagg

SEQ ID NO: 103 - DNA, 111 bp, Canis familiaris
aacctactctctgccatcaagagcccttgccaaagggagaccccagaggggaccgaggccaagccctggtacgagcccatctacctgggaggggtcttccaactggagaag

SEQ ID NO: 104 - DNA, 98 bp, Canis familiaris
tactttggaatcattgccctgtaaggggrtaggacgtccattcttgcccaaaccgaccctttgatcactcacttcctctgacccctcacccccttcag

SEQ ID NO: 105 - DNA, 47 bp, Canis familiaris
cctgagagaggatttttttawttttaatttttttaagatttatttga

SEQ ID NO: 106 - DNA, 55 bp, Canis familiaris
ttcccagatgactgagtggctgagcttsactgaaagacggagaaacagaggctca

SEQ ID NO: 107 - DNA, 56 bp, Canis familiaris
cagtctatccaagataatgtacccagcacaayaccccatgtataatggcaatgagt

SEQ ID NO: 108 - DNA, 62 bp, Canis familiaris
gccctgtggaccctctggggggggcagrgggggatgaggaagggacaccttttgttccagag

SEQ ID NO: 109 - DNA, 21 bp, Artificial
  Primer CTLA4 11R124F
ggttgcttttgcctgctaaca

SEQ ID NO: 110 - DNA, 17 bp, Artificial
  Reporter sequence CTLA4 11R124V
tttcagctggatttgaa

SEQ ID NO: 111 - DNA, 18 bp, Artificial
  Primer CTLA4 11R204F
agggcctcaggagaagct

SEQ ID NO: 112 - DNA, 19 bp, Artificial
  Reporter sequence CTLA4 11R204V
ctgtcgtattaattaactg

SEQ ID NO: 113 - DNA, 26 bp, Artificial
  Primer CTLA4 11R269F
gaggagaagaaggaggattggataag

SEQ ID NO: 114 - DNA, 17 bp, Artificial
  Reporter sequence CTLA4 11R269V
cattggaacaacatgag

SEQ ID NO: 115 - DNA, 25 bp, Artificial
  Primer CTLA4 11M291F
atgggagaaaataggcattggaaca

SEQ ID NO: 116 - DNA, 20 bp, Artificial
  Reporter sequence CTLA4 11M291V
catacctcttacatatctca

SEQ ID NO: 117 - DNA, 25 bp, Artificial
  Primer CTLA4 11R308F
atgggagaaaataggcattggaaca

SEQ ID NO: 118 - DNA, 20 bp, Artificial
  Reporter sequence CTLA4 11R308V
tcctctttttgttcaacata

SEQ ID NO: 119 - DNA, 22 bp, Artificial
  Primer CTLA4 11R364F
ggcatgtgaagaaatgctggaa

SEQ ID NO: 120 - DNA, 20 bp, Artificial
  Reporter sequence CTLA4 11R364V
ctaaaaagagaagcattagg

SEQ ID NO: 121 - DNA, 20 bp, Artificial
  Primer CTLA4 11R386F
tgctggaagccaggctaaaa

SEQ ID NO: 122 - DNA, 17 bp, Artificial
  Reporter sequence CTLA4 11R386V
ttccacaaagtgtcctc

SEQ ID NO: 123 - DNA, 25 bp, Artificial
  Primer CTLA4 11Y437F
ctgagctatatggacagtgggaaat

SEQ ID NO: 124 - DNA, 16 bp, Artificial
  Reporter sequence CTLA4 11Y437V
ctattcccgcacttta

SEQ ID NO: 125 - DNA, 25 bp, Artificial
  Primer CTLA4 11Y540F
tctcctagaagtcccttaaggcatt

SEQ ID NO: 126 - DNA, 18 bp, Artificial
  Reporter sequence CTLA4 11Y540V
cacttagattgaaagatg

SEQ ID NO: 127 - DNA, 28 bp, Artificial
  Primer CTLA4 12K291F
ctcatatggaaggtttgaatctcttgga

SEQ ID NO: 128 - DNA, 20 bp, Artificial
  Reporter sequence CTLA4 12K291V
ctgactttttttggaccgaa

SEQ ID NO: 129 - DNA, 26 bp, Artificial
  Primer CTLA4 12K375F
tgaattctttcctaatctgcaagcca

SEQ ID NO: 130 - DNA, 16 bp, Artificial
  Reporter sequence CTLA4 12K375V
aattcattgatttccc

SEQ ID NO: 131 - DNA, 27 bp, Artificial
  Primer CTLA4 12M78F
gcatatcacaggttctcaagaaatgtc

SEQ ID NO: 132 - DNA, 20 bp, Artificial
  Reporter sequence CTLA4 12M78V
atgaaaactcctcagtatta

SEQ ID NO: 133 - DNA, 30 bp, Artificial
  Primer CTLA4 12Y232F
cttggattttatgcttgaaaagttcccttt

SEQ ID NO: 134 - DNA, 19 bp, Artificial
  Reporter sequence CTLA4 12Y232V
atatgaggagagacatgtt

SEQ ID NO: 135 - DNA, 28 bp, Artificial
  Primer CTLA4 13R176F
gcagggcttttattaatgatgtctatgg

SEQ ID NO: 136 - DNA, 17 bp, Artificial
  Reporter sequence CTLA4 13R176V
tcaaggactcagaccag

SEQ ID NO: 137 - DNA, 27 bp, Artificial
  Primer CTLA4 13Y435F
agtgtttgaggttatcttttcgacgta

SEQ ID NO: 138 - DNA, 17 bp, Artificial
  Reporter sequence CTLA4 13Y435V
aaaggaagccgtgggtt

SEQ ID NO: 139 - DNA, 31 bp, Artificial
  Primer IFNg 4R430F
gtatcagtcccattttaaagatagggaaact

SEQ ID NO: 140 - DNA, 19 bp, Artificial
  Reporter sequence IFNg 4R430V
cctaactctcctatgattc

SEQ ID NO: 141 - DNA, 29 bp, Artificial
  Primer IFNg 5M509F
aggtttgagttcccttagaatttcctttt

SEQ ID NO: 142 - DNA, 17 bp, Artificial
  Reporter sequence IFNg 5M509V
accacaaaaaaattatc

SEQ ID NO: 143 - DNA, 30 bp, Artificial
  Primer IFNg 5M532F
ggtttgagttcccttagaatttcctttttt

SEQ ID NO: 144 - DNA, 19 bp, Artificial
  Reporter sequence IFNg 5M532V
tgttgagtcctcatccata

SEQ ID NO: 145 - DNA, 21 bp, Artificial
  Primer IFNg 15Y221F
agacgccacttgaatgtgtca

SEQ ID NO: 146 - DNA, 17 bp, Artificial
  Reporter sequence IFNg 15Y221V
caggacacaggtcatat

SEQ ID NO: 147 - DNA, 31 bp, Artificial
  Primer IFNg 15W376F
gactgtacccaatggaaaacaattaatttgt

SEQ ID NO: 148 - DNA, 17 bp, Artificial
  Reporter sequence IFNg 15W376V
tttgacacagcatgaat

SEQ ID NO: 149 - DNA, 17 bp, Artificial
  Primer IL-10 4Y100F
cagccgacaccagagca

SEQ ID NO: 150 - DNA, 16 bp, Artificial
  Reporter sequence IL-10 4Y100V
tcgtcctcaggtaggg

SEQ ID NO: 151 - DNA, 17 bp, Artificial
  Primer IL-10 6R426F
gctcttccgcccagtca

SEQ ID NO: 152 - DNA, 17 bp, Artificial
  Reporter sequence IL-10 6R426V
cccaggtaactctaaag

SEQ ID NO: 153 - DNA, 25 bp, Artificial
  Primer IL-12b 10R105F
tcatgaagctcacaatccagttctc

SEQ ID NO: 154 - DNA, 18 bp, Artificial
  Reporter sequence IL-12b 10R105V
caactctacaatataaac

SEQ ID NO: 155 - DNA, 33 bp, Artificial
  Primer IL-12B 12Y142F
gaatttttgttcttttcaaatccagaatccaaa

SEQ ID NO: 156 - DNA, 16 bp, Artificial
  Reporter sequence IL-12B 12Y142V
ccagaatgattctttg

SEQ ID NO: 157 - DNA, 22 bp, Artificial
  Primer IL-6 18R120F
tggctgaagcacacaacaattc

SEQ ID NO: 158 - DNA, 15 bp, Artificial
  Reporter sequence IL-6 18R120V
catcctgcagagtct

SEQ ID NO: 159 - DNA, 29 bp, Artificial
  Primer RANTES 13W74F
agtcatattctccctgtttcatagatgga

SEQ ID NO: 160 - DNA, 19 bp, Artificial
  Reporter sequence RANTES 13W74V
agaggatttttttaatttt

SEQ ID NO: 161 - DNA, 27 bp, Artificial
  Primer RANTES 13S358F
tgctctgcatgtaccatgtcatttaat

SEQ ID NO: 162 - DNA, 16 bp, Artificial
  Reporter sequence RANTES 13S358V
ctttcagtgaagctca

SEQ ID NO: 163 - DNA, 26 bp, Artificial
  Primer RANTES 17Y105F
cagtttcagccaaagaaggataacag

SEQ ID NO: 164 - DNA, 15 bp, Artificial
  Reporter sequence RANTES 17Y105V
cagcacaacacccca

SEQ ID NO: 165 - DNA, 17 bp, Artificial
  Primer RANTES 17R307F
ccctgtggaccctctgg

SEQ ID NO: 166 - DNA, 16 bp, Artificial
  Reporter sequence RANTES 17R307V
ctcatccccctctgcc

SEQ ID NO: 167 - DNA, 23 bp, Artificial
  Primer RANTES 17M347F
tgaggaagggacaccttttgttc

SEQ ID NO: 168 - DNA, 16 bp, Artificial
  Reporter sequence RANTES 17M347V
cagagccagtacccca

SEQ ID NO: 169 - DNA, 21 bp, Artificial
  Primer CTLA4 11R124R
cccctccccccaaccttatat

SEQ ID NO: 170 - DNA, 15 bp, Artificial
  Reporter sequence CTLA4 11R124M
tcagctgggtttgaa

SEQ ID NO: 171 - DNA, 28 bp, Artificial
  Primer CTLA4 11R204R
tctcccattatcttatccaatcctcctt

SEQ ID NO: 172 - DNA, 19 bp, Artificial
  Reporter sequence CTLA4 11R204M
ctgtcgtattagttaactg

SEQ ID NO: 173 - DNA, 23 bp, Artificial
  Primer CTLA4 11R269R
ggcttccagcatttcttcacatg

SEQ ID NO: 174 - DNA, 17 bp, Artificial
  Reporter sequence CTLA4 11R269M
cattggaacagcatgag

SEQ ID NO: 175 - DNA, 23 bp, Artificial
  Primer CTLA4 11M291R
ggcttccagcatttcttcacatg

SEQ ID NO: 176 - DNA, 16 bp, Artificial
  Reporter sequence CTLA4 11M291M
cctcttacagatctca

SEQ ID NO: 177 - DNA, 23 bp, Artificial
  Primer CTLA4 11R308R
ggcttccagcatttcttcacatg

SEQ ID NO: 178 - DNA, 18 bp, Artificial
  Reporter sequence CTLA4 11R308M
ctctttttgtccaacata

SEQ ID NO: 179 - DNA, 25 bp, Artificial
  Primer CTLA4 11R364R
gtccatatagctcagcacagtagag

SEQ ID NO: 180 - DNA, 17 bp, Artificial
  Reporter sequence CTLA4 11R364M
aaaagagaggcattagg

SEQ ID NO: 181 - DNA, 24 bp, Artificial
  Primer CTLA4 11R386R
acaggcaaacagacagttacaaca

SEQ ID NO: 182 - DNA, 15 bp, Artificial
  Reporter sequence CTLA4 11R386M
ccacagagtgtcctc

SEQ ID NO: 183 - DNA, 24 bp, Artificial
  Primer CTLA4 11Y437R
acaggcaaacagacagttacaaca

SEQ ID NO: 184 - DNA, 17 bp, Artificial
  Reporter sequence CTLA4 11Y437M
cctattcccacacttta

SEQ ID NO: 185 - DNA, 24 bp, Artificial
  Primer CTLA4 11Y540R
gagaacctgtgatatgccagtgat

SEQ ID NO: 186 - DNA, 18 bp, Artificial
  Reporter sequence CTLA4 11Y540M
cacttagattaaaagatg

SEQ ID NO: 187 - DNA, 31 bp, Artificial
  Primer CTLA4 12K291R
tcaggtattcttaaaagccttaacaaagaca

SEQ ID NO: 188 - DNA, 20 bp, Artificial
  Reporter sequence CTLA4 12K291M
ctgactttttttggacctaa

SEQ ID NO: 189 - DNA, 27 bp, Artificial
  Primer CTLA4 12K375R
agctccatttagcattttgttgagaga

SEQ ID NO: 190 - DNA, 18 bp, Artificial
  Reporter sequence CTLA4 12K375M
caaattcatttatttccc

SEQ ID NO: 191 - DNA, 26 bp, Artificial
  Primer CTLA4 12M78R
aggaccagtgttcatactgtaagaga

SEQ ID NO: 192 - DNA, 20 bp, Artificial
  Reporter sequence CTLA4 12M78M
atgaaaactcctccgtatta

SEQ ID NO: 193 - DNA, 25 bp, Artificial
  Primer CTLA4 12Y232R
aagtcagccaaaatgatccaagaga

SEQ ID NO: 194 - DNA, 19 bp, Artificial
  Reporter sequence CTLA4 12Y232M
atatgaggagaaacatgtt

SEQ ID NO: 195 - DNA, 22 bp, Artificial
  Primer CTLA4 13R176R
cacatctccaaagctgctaagc

SEQ ID NO: 196 - DNA, 15 bp, Artificial
  Reporter sequence CTLA4 13R176M
aagggctcagaccag

SEQ ID NO: 197 - DNA, 25 bp, Artificial
  Primer CTLA4 13Y435R
gcacctgaatagaaagcctttttgt

SEQ ID NO: 198 - DNA, 17 bp, Artificial
  Reporter sequence CTLA4 13Y435M
aaaggaagccatgggtt

SEQ ID NO: 199 - DNA, 24 bp, Artificial
  Primer IFNg 4R430R
ggctatgtgattctgaggaagcat

SEQ ID NO: 200 - DNA, 17 bp, Artificial
  Reporter sequence IFNg 4R430M
taactctcccatgattc

SEQ ID NO: 201 - DNA, 25 bp, Artificial
  Primer IFNg 5M509R
acctccatcctttgcctttgtaaat

SEQ ID NO: 202 - DNA, 15 bp, Artificial
  Reporter sequence IFNg 5M509M
cacaaacaaattatc

SEQ ID NO: 203 - DNA, 25 bp, Artificial
  Primer IFNg 5M532R
acctccatcctttgcctttgtaaat

SEQ ID NO: 204 - DNA, 17 bp, Artificial
  Reporter sequence IFNg 5M532M
ttgagtcctcagccata

SEQ ID NO: 205 - DNA, 26 bp, Artificial
  Primer IFNg 15Y221R
gggtacagtcatagttgtcagtggta

SEQ ID NO: 206 - DNA, 17 bp, Artificial
  Reporter sequence IFNg 15Y221M
caggacacaagtcatat

SEQ ID NO: 207 - DNA, 35 bp, Artificial
  Primer IFNg 15W376R
aactcattagagtatatagttcatttaccccagaa

SEQ ID NO: 208 - DNA, 16 bp, Artificial
  Reporter sequence IFNg 15W376M
ttgacactgcatgaat

SEQ ID NO: 209 - DNA, 17 bp, Artificial
  Primer IL-10 4Y100R
aggctggctgggaagtg

SEQ ID NO: 210 - DNA, 16 bp, Artificial
  Reporter sequence IL-10 4Y100M
tcgtcctcaagtaggg

SEQ ID NO: 211 - DNA, 25 bp, Artificial
  Primer IL-10 6R426R
cctcctccaagtaaaactggatcat

SEQ ID NO: 212 - DNA, 17 bp, Artificial
  Reporter sequence IL-10 6R426M
cccaggtaaccctaaag

SEQ ID NO: 213 - DNA, 22 bp, Artificial
  Primer IL-12b 10R105R
caggtgaggaccaccatttctc

SEQ ID NO: 214 - DNA, 18 bp, Artificial
  Reporter sequence IL-12b 10R105M
caactctacaacataaac

SEQ ID NO: 215 - DNA, 19 bp, Artificial
  Primer IL-12B 12Y142R
gccaccagcatgtgaaacg

SEQ ID NO: 216 - DNA, 17 bp, Artificial
  Reporter sequence IL-12B 12Y142M
tccagaataattctttg

SEQ ID NO: 217 - DNA, 23 bp, Artificial
  Primer IL-6 18R120R
cagactgaactgcaggaaatcct

SEQ ID NO: 218 - DNA, 15 bp, Artificial
  Reporter sequence IL-6 18R120M
catcctgcggagtct

SEQ ID NO: 219 - DNA, 25 bp, Artificial
  Primer RANTES 13W74R
ccctcccctctattctctctcaaat

SEQ ID NO: 220 - DNA, 19 bp, Artificial
  Reporter sequence RANTES 13W74M
agaggatttttttattttt

SEQ ID NO: 221 - DNA, 22 bp, Artificial
  Primer RANTES 13S358R
ctcctctgagcctctgtttctc

SEQ ID NO: 222 - DNA, 16 bp, Artificial
  Reporter sequence RANTES 13S358M
ctttcagtcaagctca

SEQ ID NO: 223 - DNA, 25 bp, Artificial
  Primer RANTES 17Y105R
gtagactcctgtactcattgccatt

SEQ ID NO: 224 - DNA, 15 bp, Artificial
  Reporter sequence RANTES 17Y105M
cagcacaatacccca

SEQ ID NO: 225 - DNA, 21 bp, Artificial
  Primer RANTES 17R307R
actggctctggaacaaaaggt

SEQ ID NO: 226 - DNA, 15 bp, Artificial
  Reporter sequence RANTES 17R307M
tcatccccccctgcc

SEQ ID NO: 227 - DNA, 23 bp, Artificial
  Primer RANTES 17M347R
ggagtggatagggtaggctctta

SEQ ID NO: 228 - DNA, 15 bp, Artificial
  Reporter sequence RANTES 17M347M
agagccagtccccca

SEQ ID NO: 229 - DNA, 30 bp, Artificial
  Primer IL-4_7S246 2nd PCRP
acgttggatgaagaatcaggtgacaggctc

SEQ ID NO: 230 - DNA, 30 bp, Artificial
  Primer  IL-4_7S246 1st PCRP
acgttggatgggaagagctcagagtagatg

SEQ ID NO: 231 - DNA, 30 bp, Artificial
  Primer IL-12B_10R105 2nd PCRP
acgttggatgtgaggaccaccatttctccg

SEQ ID NO: 232 - DNA, 30 bp, Artificial
  Primer IL-12B_10R105 1st PCRP
acgttggatgacaatccagttctccactcc

SEQ ID NO: 233 - DNA, 30 bp, Artificial
  Primer IL-12B_02M407 2nd PCRP
acgttggatgccacactttgagaaccactg

SEQ ID NO: 234 - DNA, 30 bp, Artificial
  Primer IL-12B_02M407 1st PCRP
acgttggatggtcttctcccaaagaacctc

SEQ ID NO: 235 - DNA, 30 bp, Artificial
  Primer IL-12B_03Y82 2nd PCRP
acgttggatgtaacaaggcttccaggttac

SEQ ID NO: 236 - DNA, 30 bp, Artificial
  Primer IL-12B_03Y82 1st PCRP
acgttggatggctccaaactcaaaggttac

SEQ ID NO: 237 - DNA, 30 bp, Artificial
  Primer IL-12B_02Y190 2nd PCRP
acgttggatgatgctctctgagatggatgg

SEQ ID NO: 238 - DNA, 30 bp, Artificial
  Primer IL-12B_02Y190 1st PCRP
acgttggatgatgtgaaaactgtaccctac

SEQ ID NO: 239 - DNA, 30 bp, Artificial
  Primer IL-12B_01Y90 2nd PCRP
acgttggatgcagccaggcacgacttttta

SEQ ID NO: 240 - DNA, 30 bp, Artificial
  Primer IL-12B_01Y90 1st PCRP
acgttggatgatgtcagcttgtaccaaggg

SEQ ID NO: 241 - DNA, 30 bp, Artificial
  Primer TNFexon4aAB 2nd PCRP
acgttggatgactcggcaaagtccagatag

SEQ ID NO: 242 - DNA, 30 bp, Artificial
  Primer TNFexon4aAB 1st PCRP
acgttggatgggtcttccaactggagaagg

SEQ ID NO: 243 - DNA, 30 bp, Artificial
  Primer IL-4_8R4582nd PCRP
acgttggatgctggtgcccagaaaattgag

SEQ ID NO: 244 - DNA, 30 bp, Artificial
  Primer IL-4_8R4581st PCRP
acgttggatggaactcctgatcttctgctc

SEQ ID NO: 245 - DNA, 30 bp, Artificial
  Primer IL-10_1R117 2nd PCRP
acgttggatggtcccttgatgtaccttgac

SEQ ID NO: 246 - DNA, 30 bp, Artificial
  Primer IL-10_1R117 1st PCRP
acgttggatgtgctcttcctagttactgtc

SEQ ID NO: 247 - DNA, 30 bp, Artificial
  Primer IL-10_1R218 2nd PCRP
acgttggatgcgccctctcctttccttatt

SEQ ID NO: 248 - DNA, 30 bp, Artificial
  Primer IL-10_1R218 1st PCRP
acgttggatgtgtgtgtgtgtttgagggtg

SEQ ID NO: 249 - DNA, 30 bp, Artificial
  Primer IL-4_25Y336 2nd PCRP
acgttggatggaattactggatcatgtagc

SEQ ID NO: 250 - DNA, 30 bp, Artificial
  Primer IL-4_25Y336 1st PCRP
acgttggatgaaactggtgcagccactatg

SEQ ID NO: 251 - DNA, 29 bp, Artificial
  Primer IL-10_4Y100 2nd PCRP
acgttggatgactgctctgttgctgcctg

SEQ ID NO: 252 - DNA, 29 bp, Artificial
  Primer IL-10_4Y100 1st PCRP
acgttggatgtgggaagtgggtgcagtcg

SEQ ID NO: 253 - DNA, 31 bp, Artificial
  Primer IL-12B_12Y142 2nd PCRP
acgttggatggatctttctgaaatgtgaggc

SEQ ID NO: 254 - DNA, 30 bp, Artificial
  Primer IL-12B_12Y142 1st PCRP
acgttggatgcaaatcagtactgattgccg

SEQ ID NO: 255 - DNA, 30 bp, Artificial
  Primer TNF10252 2nd PCRP
acgttggatgatcaagagcccttgccaaag

SEQ ID NO: 256 - DNA, 30 bp, Artificial
  Primer TNF10252 1st PCRP
acgttggatgttctccagttggaagacccc

SEQ ID NO: 257 - DNA, 30 bp, Artificial
  Primer TNF7178 2nd PCRP
acgttggatgatctgcaccttcaacgaagc

SEQ ID NO: 258 - DNA, 30 bp, Artificial
  Primer TNF7178 1st PCRP
acgttggatgaaaattctcccctcccagac

SEQ ID NO: 259 - DNA, 30 bp, Artificial
  Primer IL-12B_03R196 2nd PCRP
acgttggatgtggtggtgggagacaattag

SEQ ID NO: 260 - DNA, 30 bp, Artificial
  Primer IL-12B_03R196 1st PCRP
acgttggatgggagagaaactaaacctggc

SEQ ID NO: 261 - DNA, 30 bp, Artificial
  Primer TNF10411 2nd PCRP
acgttggatgagtgagtgatcaaagggtcg

SEQ ID NO: 262 - DNA, 30 bp, Artificial
  Primer TNF10411 1st PCRP
acgttggatgggcaggtgtactttggaatc

SEQ ID NO: 263 - DNA, 30 bp, Artificial
  Primer IL-10_14R553 2nd PCRP
acgttggatgacagccgatgagatgttgac

SEQ ID NO: 264 - DNA, 30 bp, Artificial
  Primer IL-10_14R553 1st PCRP
acgttggatgaatcccataccctatggctg

SEQ ID NO: 265 - DNA, 30 bp, Artificial
  Primer IL-10_11R124 2nd PCRP
acgttggatgtcgctagccacgctttttag

SEQ ID NO: 266 - DNA, 30 bp, Artificial
  Primer IL-10_11R124 1st PCRP
acgttggatgtgaaggatggacccaggcaa

SEQ ID NO: 267 - DNA, 30 bp, Artificial
  Primer IL-6_20R191 2nd PCRP
acgttggatgcttctagctgggtgactttg

SEQ ID NO: 268 - DNA, 30 bp, Artificial
  Primer IL-6_20R191 1st PCRP
acgttggatgtatgatgctcaatcccagcc

SEQ ID NO: 269 - DNA, 30 bp, Artificial
  Primer IL-10_9R210 2nd PCRP
acgttggatgaagtgttaatgccacggctc

SEQ ID NO: 270 - DNA, 30 bp, Artificial
  Primer IL-10_9R210 1st PCRP
acgttggatggagtctgggccctttttcag

SEQ ID NO: 271 - DNA, 29 bp, Artificial
  Primer IL-10_10S308 2nd PCRP
acgttggatgcaccctcttcccagaacag

SEQ ID NO: 272 - DNA, 28 bp, Artificial
  Primer IL-10_10S308 1st PCRP
acgttggatggggagcaggccctgcccg

SEQ ID NO: 273 - DNA, 30 bp, Artificial
  Primer TNFexon1AB 2nd PCRP
acgttggatgttctgcctcagcctcttctc

SEQ ID NO: 274 - DNA, 30 bp, Artificial
  Primer TNFexon1AB 1st PCRP
acgttggatgatcactccaaagtgcagcag

SEQ ID NO: 275 - DNA, 30 bp, Artificial
  Primer IL-4_2M3512nd PCRP
acgttggatggtgagggttcacttcatttg

SEQ ID NO: 276 - DNA, 31 bp, Artificial
  Primer IL-4_2M351 1st PCRP
acgttggatggcacaggtaatacaagatctg

SEQ ID NO: 277 - DNA, 30 bp, Artificial
  Primer IL-12B_02Y146 2nd PCRP
acgttggatgtctccatccatctcagagag

SEQ ID NO: 278 - DNA, 29 bp, Artificial
  Primer IL-12B_02Y146 1st PCRP
acgttggatgcttcttatgatttagtcag

SEQ ID NO: 279 - DNA, 30 bp, Artificial
  Primer IL-6_8R2892nd PCRP
acgttggatgttaccagattagcgggctag

SEQ ID NO: 280 - DNA, 30 bp, Artificial
  Primer IL-6_8R2891st PCRP
acgttggatggaagctcaggtctaaacgtc

SEQ ID NO: 281 - DNA, 30 bp, Artificial
  Primer IL1a10084 2nd PCRP
acgttggatggaatgacttagcccacactc

SEQ ID NO: 282 - DNA, 30 bp, Artificial
  Primer IL1a10084 1st PCRP
acgttggatgggaggcagatacatatgcag

SEQ ID NO: 283 - DNA, 30 bp, Artificial
  Primer IL-6_7S1662nd PCRP
acgttggatgtgttttgagtccagaggtgc

SEQ ID NO: 284 - DNA, 30 bp, Artificial
  Primer IL-6_7S166 1st PCRP
acgttggatgaagaaaacctagggcaagcg

SEQ ID NO: 285 - DNA, 30 bp, Artificial
  Primer IL-4_22Y152 2nd PCRP
acgttggatgctctccctactgatttcctc

SEQ ID NO: 286 - DNA, 30 bp, Artificial
  Primer IL-4_22Y152 1st PCRP
acgttggatgaatatggttgcagggccttc

SEQ ID NO: 287 - DNA, 30 bp, Artificial
  Primer IL-6_20R240 2nd PCRP
acgttggatgtcacccagctagaaggtaag

SEQ ID NO: 288 - DNA, 30 bp, Artificial
  Primer IL-6_20R240 1st PCRP
acgttggatggggaccctaaaggttaagag

SEQ ID NO: 289 - DNA, 30 bp, Artificial
  Primer IL-6_7R485 2nd PCRP
acgttggatgactctcttgctcacctcttc

SEQ ID NO: 290 - DNA, 30 bp, Artificial
  Primer IL-6_7R485 1st PCRP
acgttggatgagatccaagtcttcaccagg

SEQ ID NO: 291 - DNA, 30 bp, Artificial
  Primer IL-6_18R120 2nd PCRP
acgttggatgctgaactgcaggaaatcctc

SEQ ID NO: 292 - DNA, 30 bp, Artificial
  Primer IL-6_18R120 1st PCRP
acgttggatgtatcttgcagtcgcaggatg

SEQ ID NO: 293 - DNA, 30 bp, Artificial
  Primer IL-6_20R412 2nd PCRP
acgttggatgttggaagtgcacattgctag

SEQ ID NO: 294 - DNA, 30 bp, Artificial
  Primer IL-6_20R412 1st PCRP
acgttggatgagggaatgcatgtaaagatg

SEQ ID NO: 295 - DNA, 30 bp, Artificial; identical to SEQ ID NO: 240
  Primer IL-12B_01M115 2nd PCRP

SEQ ID NO: 296 - DNA, 30 bp, Artificial
  Primer IL-12B_01M115 1st PCRP
acgttggatgggcacgactttttaccctac

SEQ ID NO: 297 - DNA, 30 bp, Artificial
  Primer TNF6547 2nd PCRP
acgttggatgcagaatggaggcaaaatggg

SEQ ID NO: 298 - DNA, 30 bp, Artificial
  Primer TNF6547 1st PCRP
acgttggatgtgtcttctttggagccttcg

SEQ ID NO: 299 - DNA, 30 bp, Artificial
  Primer IL-4_1K110 2nd PCRP
acgttggatggccacttctggatgtttcat

SEQ ID NO: 300 - DNA, 30 bp, Artificial
  Primer IL-4_1K110 1st PCRP
acgttggatgcgctacaatatggatgaacc

SEQ ID NO: 301 - DNA, 30 bp, Artificial
  Primer IL1a11235 2nd PCRP
acgttggatgaccgtgtgtgttaccaaagc

SEQ ID NO: 302 - DNA, 30 bp, Artificial
  Primer IL1a11235 1st PCRP
acgttggatgctgtcaaacaagataatgag

SEQ ID NO: 303 - DNA, 30 bp, Artificial
  Primer IL-10_13Y85 2nd PCRP
acgttggatgtacagacgccatagtcttcc

SEQ ID NO: 304 - DNA, 30 bp, Artificial
  Primer IL-10_13Y85 1st PCRP
acgttggatgccttagtcttgaaaaccagc

SEQ ID NO: 305 - DNA, 30 bp, Artificial
  Primer IL-6_6R431 2nd PCRP
acgttggatgagcaatcccacactacagag

SEQ ID NO: 306 - DNA, 30 bp, Artificial
  Primer IL-6_6R4311st PCRP
acgttggatgctctcctgcgctgaatgaag

SEQ ID NO: 307 - DNA, 30 bp, Artificial
  Primer IL1aE7x221 2nd PCRP
acgttggatgtacatagtacacgtggactg

SEQ ID NO: 308 - DNA, 30 bp, Artificial
  Primer IL1aE7x221 1st PCRP
acgttggatgctttcggttactggaaaccc

SEQ ID NO: 309 - DNA, 30 bp, Artificial
  Primer IL-4_12M397 2nd PCRP
acgttggatgctggatattggtgctttggg

SEQ ID NO: 310 - DNA, 30 bp, Artificial
  Primer IL-4_12M397 1st PCRP
acgttggatgctttgcagacacttgccacc

SEQ ID NO: 311 - DNA, 30 bp, Artificial
  Primer IL-10_6R426 2nd PCRP
acgttggatgactggatcatctccgacagg

SEQ ID NO: 312 - DNA, 29 bp, Artificial
  Primer IL-10_6R426 1st PCRP
acgttggatgcagctcttccgcccagtca

SEQ ID NO: 313 - DNA, 30 bp, Artificial
  Primer TNF8647 2nd PCRP
acgttggatgctaatatacaaggccccagg

SEQ ID NO: 314 - DNA, 30 bp, Artificial
  Primer TNF8647 1st PCRP
acgttggatgctttcagtgctcatggtgtg

SEQ ID NO: 315 - DNA, 30 bp, Artificial
  Primer IL-4_13S972nd PCRP
acgttggatgagatcagaggaagcttctgg

SEQ ID NO: 316 - DNA, 30 bp, Artificial
  Primer IL-4_13S971st PCRP
acgttggatgctatacctcctaggccaaag

SEQ ID NO: 317 - DNA, 30 bp, Artificial
  Primer IL-10_6Y135 2nd PCRP
acgttggatggcagcaaatgaaggacaagc

SEQ ID NO: 318 - DNA, 30 bp, Artificial
  Primer IL-10_6Y135 1st PCRP
acgttggatggctctcaccttaaagtcctc

SEQ ID NO: 319 - DNA, 30 bp, Artificial
  Primer IL-12B_02W232 2nd PCRP
acgttggatgttactatccagggtttgtgc

SEQ ID NO: 320 - DNA, 29 bp, Artificial
  Primer IL-12B_02W232 1st PCRP
acgttggatgcaggatgagatgaaatgat

SEQ ID NO: 321 - DNA, 30 bp, Artificial; identical to SEQ ID NO: 246
  Primer IL-10_1R105 2nd PCRP

SEQ ID NO: 322 - DNA, 30 bp, Artificial; identical to SEQ ID NO: 245
  Primer IL-10_1R105 1st PCRP

SEQ ID NO: 323 - DNA, 30 bp, Artificial
  Primer TNF9585 2nd PCRP
acgttggatgttcaggcacttgtttgaggg

SEQ ID NO: 324 - DNA, 30 bp, Artificial
  Primer TNF9585 1st PCRP
acgttggatgggtgagatccttaagcttcc

SEQ ID NO: 325 - DNA, 30 bp, Artificial
  Primer IL-10_2R420 2nd PCRP
acgttggatgaataattggatcccctcccc

SEQ ID NO: 326 - DNA, 30 bp, Artificial
  Primer IL-10_2R420 1st PCRP
acgttggatggaaactgaggctcttcccag

SEQ ID NO: 327 - DNA, 30 bp, Artificial
  Primer TNF9367 2nd PCRP
acgttggatgggatggatgggagagaaaac

SEQ ID NO: 328 - DNA, 30 bp, Artificial
  Primer TNF9367 1st PCRP
acgttggatgaggaggtttagcatcagagc

SEQ ID NO: 329 - DNA, 30 bp, Artificial
  Primer IL-2_12Y206 2nd PCRP
acgttggatggaattcttgtgttcactgag

SEQ ID NO: 330 - DNA, 31 bp, Artificial
  Primer IL-2_12Y206 1st PCRP
acgttggatggttgatacaagtgatgatagc

SEQ ID NO: 331 - DNA, 30 bp, Artificial
  Primer TNF10513 2nd PCRP
acgttggatgctcacatccctggatcttag

SEQ ID NO: 332 - DNA, 30 bp, Artificial
  Primer TNF10513 1st PCRP
acgttggatgcccttcaggcttagaaagag

SEQ ID NO: 333 - DNA, 30 bp, Artificial
  Primer IL1a12227 2nd PCRP
acgttggatgatccttgtgacagaaagcag

SEQ ID NO: 334 - DNA, 31 bp, Artificial
  Primer IL1a12227 1st PCRP
acgttggatggtaacttacaaaggagcatag

SEQ ID NO: 335 - DNA, 30 bp, Artificial
  Primer IL-6_10Y257 2nd PCRP
acgttggatgtttgcagaggtgagtggtag

SEQ ID NO: 336 - DNA, 30 bp, Artificial
  Primer IL-6_10Y257 1st PCRP
acgttggatgatggctactgctttccctac

SEQ ID NO: 337 - DNA, 30 bp, Artificial
  Primer IL-10_3M171 2nd PCRP
acgttggatggttcaccccaggaaatcaac

SEQ ID NO: 338 - DNA, 30 bp, Artificial
  Primer IL-10_3M171 1st PCRP
acgttggatgattttaggatgagctacctc

SEQ ID NO: 339 - DNA, 30 bp, Artificial
  Primer IL1aE7x2552nd PCRP
acgttggatggccttgactctggagtctat

SEQ ID NO: 340 - DNA, 31 bp, Artificial
  Primer IL1aE7x2551st PCRP
acgttggatggcaagtgactatgagtaaagg

SEQ ID NO: 341 - DNA, 30 bp, Artificial
  Primer IL-6_8W328 2nd PCRP
acgttggatgggtgagaagctaaggctatg

SEQ ID NO: 342 - DNA, 30 bp, Artificial
  Primer IL-6_8W328 1st PCRP
acgttggatgaatgctaaatcctagcccgc

SEQ ID NO: 343 - DNA, 30 bp, Artificial
  Primer 12B_03R462 2nd PCRP
acgttggatggcaggaacatgacttattgg

SEQ ID NO: 344 - DNA, 30 bp, Artificial
  Primer 12B_03R462 1st PCRP
acgttggatgtctcgctcagagccttttac

SEQ ID NO: 345 - DNA, 30 bp, Artificial
  Primer IL1a8619 2nd PCRP
acgttggatgtattggcatcttgaggctgg

SEQ ID NO: 346 - DNA, 30 bp, Artificial
  Primer IL1a8619 1st PCRP
acgttggatgccaatcaggaaaccttcaac

SEQ ID NO: 347 - DNA, 30 bp, Artificial
  Primer IL-10_1K362 2nd PCRP
acgttggatgccagtcttcatggaatcctg

SEQ ID NO: 348 - DNA, 30 bp, Artificial
  Primer IL-10_1K362 1st PCRP
acgttggatgctgtggttggacacttaagc

SEQ ID NO: 349 - DNA, 30 bp, Artificial
  Primer IL-6_6K372 2nd PCRP
acgttggatgtaaaccactaagccaccagg

SEQ ID NO: 350 - DNA, 30 bp, Artificial
  Primer IL-6_6K3721st PCRP
acgttggatgaaaagcctctgtagtgtggg

(19) United States
(12) Patent Application Publication
(10) Pub. No.: US 2009/0308324 A1
(43) Pub. Date: Dec. 17, 2009

(54) DIABETES TESTS

(75) Inventors: Neale Fretwell, Melton Mowbray (GB); Christopher Andrew Jones, Nottingham (GB); Andrea Dawn Short, Manchester (GB); William Ernest Royce Ollier, Manchester (GB); Lorna Jane Kennedy, Manchester (GB)

Correspondence Address: FULBRIGHT & JAWORSKI, LLP, 1301 MCKINNEY, SUITE 5100, HOUSTON, TX, 77010-3095, US

(73) Assignee: MARS INCORPORATED, McLean, VA (US)

(21) Appl. No.: 12/375,928

(22) PCT Filed: Aug. 1, 2007
(86) PCT No.: PCT/GB07/02934

§ 371 Date: Jan. 30, 2009

(30) Foreign Application Priority Data
Aug. 1, 2006 (GB) 0615300.1

(51) Int. Cl. A01K 67/02; C12Q 1/68; C07H 21/04; A23K 1/18; A61K 38/28; G06F 17/30; G06N 5/02; G06F 17/50

(52) U.S. Cl. 119/174; 435/6; 536/23.1; 426/2; 514/3; 707/104.1; 706/54; 707/E17.044; 700/107

(57) ABSTRACT

A method for diagnosing susceptibility to diabetes in a dog, the method comprising: (a) (i) detecting in a sample from the dog the presence or absence of a genotype in any one of the following immune system genes: CTLA-4, IGF-2, IL-1α, IL-4, IL-6, IL-1O, IL-12β, IFNγ, PTPN3, PTPN15, PTPN22, TNF, or RANTES; and/or (ii) determining in a sample from the dog whether a genotype identified in Table 1 or 3A, or a genotype in linkage disequilibrium with said genotype identified in Table 1 or 3 A, is present in an insulin or IGF gene of the dog; and/or (iii) determining in a sample from the dog whether a genotype identified in Table 2 or 3B, or a genotype in linkage disequilibrium with said genotype identified in Table 2 or 3B, is absent in an insulin or IGF gene of the dog; and (b) thereby diagnosing whether the dog is susceptible to diabetes.

### FIELD OF THE INVENTION

The present invention relates to the diagnosis and treatment of diabetes in dogs.

### BACKGROUND OF THE INVENTION

Diabetes is a significant source of morbidity in dogs. It is one of the most common endocrine disorders of dogs. The prevalence of canine diabetes in the UK is around 1 in 500 dogs and disease is typically seen in middle-aged animals between 5 and 12 years of age. Clinical signs include polydipsia, polyuria and weight loss.

Canine diabetes is not easily classified, although there are clear similarities and differences between the human and canine diseases. There is no evidence of a canine equivalent to type 2 diabetes, despite obesity being as much a problem in pet dogs as it is in their owners. The disease can be broadly divided into insulin deficiency diabetes (IDD) and insulin resistance diabetes (IRD). IDD is the most common type, although the underlying cause for the pancreatic beta cell loss is currently unknown. The commonest reason for IRD is dioestrus diabetes in female dogs, which is similar to human gestational diabetes.

### SUMMARY OF THE INVENTION

The present inventors have identified an array of genotype markers in dogs which may be used to diagnose diabetes. Accordingly, the invention provides a method for diagnosing susceptibility to diabetes in a dog, the method comprising:
(a) (i) detecting in a sample from the dog the presence or absence of a genotype in any one of the following immune system genes: CTLA-4, IGF-2, IL-1α, IL-4, IL-6, IL-10, IL-12β, IFNγ, PTPN3, PTPN15, PTPN22, TNF, or RANTES; and/or

(ii) determining in a sample from the dog whether a genotype identified in Table 1 or 3A, or a genotype in linkage disequilibrium with said genotype identified in Table 1 or 3A, is present in an insulin or IGF gene of the dog; and/or

(iii) determining in a sample from the dog whether a genotype identified in Table 2 or 3B, or a genotype in linkage disequilibrium with said genotype identified in Table 2 or 3B, is absent in an insulin or IGF gene of the dog; and
(b) thereby diagnosing whether the dog is susceptible to diabetes.

The invention further provides:
a probe or primer which is capable of detecting any of the genotypes;
a kit for carrying out the method of the invention comprising a probe or primer which is capable of detecting any of the genotypes;
a method of preparing customised food for an dog which is susceptible to diabetes, the method comprising:

(a) determining whether the dog is susceptible to diabetes by a method of the invention; and

(b) preparing food suitable for the dog;
a database comprising information relating to genotypes and optionally their association with diabetes.

### BRIEF DESCRIPTION OF THE SEQUENCES

EQ ID NOs: 1 to 108 show the polynucleotide sequences encompassing the SNPs in Tables 1, 2, 3, 5 and 6. The remaining SEQ ID NOs show the primer and probe sequences in Tables 8 and 9.

### BRIEF DESCRIPTION OF THE DRAWINGS

FIGS. 1 to 10 show haplotype frequency for cases and controls stratified into low, neutral, moderate and high risk categories of breeds for CTLA4; IGF INS; PTPN22; IFNα; IL-4; IL-10; IL-6; IL-12β; TNFα; and IL-1α respectively.

FIG. 11 illustrates schematically an embodiment of functional components arranged to carry out a method of the present invention.

### DETAILED DESCRIPTION OF THE INVENTION

The present invention provides a method for determining susceptibility to diabetes in a dog. Susceptibility to diabetes means that there is a likelihood that a dog will develop or already has diabetes. A dog that is susceptible or predisposed to the condition may have a greater than 60% chance of demonstrating symptoms that are associated with the condition. Accordingly, a dog that is susceptible may have a greater than 70%, 80% or 90% chance of exhibiting symptoms of the condition at some stage in the dog's life. For example, in a sample of 100 dogs that are diagnosed as susceptible, at least 60, at least 70, at least 80, or at least 90 of the dogs will display symptoms of the condition. In a preferred embodiment, all dogs that are diagnosed as susceptible to atopic dermatitis will display symptoms of the condition.

The diabetes condition is normally one which is caused, at least partially, by an autoimmune mechanism. In one embodiment the dog which is tested does not have any disease symptoms and/or is a healthy dog.

The dog tested is typically a companion dog or pet. The dog may be of any breed, or may be a mixed or crossbred dog, or an outbred dog (mongrel).

The dog may be of any of the breeds mentioned herein, for example in Tables 1, 2 or 4. One or both of the parents of the dog may be any of the breeds mentioned in Tables 1, 2 or 4 and/or the same breed. One, two, three or four of the grandparents of the dog may be any of the breeds mentioned in Tables 1, 2 or 4 and/or the same breed. Preferably the dog to be tested is a pure breed. However, in one embodiment, the dog to be tested may have at least 50% of any of the breeds mentioned herein. In another embodiment, the dog may have at least 75% of any of the breeds mentioned herein in its genetic breed background. Thus, at least 50% or at least 75% of its genome may be derived from any of the breeds mentioned herein. The genetic breed background of a dog may be determined by detecting the presence or absence of two or more breed-specific SNP markers in the dog.

A dog to be tested using the method of the invention may be tested for genetic breed inheritance of any of the breeds mentioned in Tables 1, 2 or 4. This could be done, for example, by analysing a sample of DNA from the dog and detecting the presence or absence of genetic markers that are inherited in the particular breed. Such markers may be single nucleotide polymorphisms (SNPs) or microsatellites, tested singly or in combination. Alternatively, the dog may not need to be tested for a particular dog breed inheritance because it is suspected of having a particular breed inheritance for example by the dog owner or veterinarian. This could be for example because of knowledge of the dog's ancestry or because of its appearance.

The dog to be tested may be of any age. Preferably the dog is from 0 to 10 years old, for example from 0 to 5 years old, from 0 to 3 years old or from 0 to 2 years old. When the method of the invention is carried out on a sample from the dog, the sample may have been taken from a dog within any of these age ranges. The dog may be tested by the method of the invention before any symptoms of diabetes are apparent.

### Detection of Genotypes

As mentioned above, in the detection method of the invention one or more genotypes may be typed in particular genes. The particular genes are the following immune system genes: CTLA-4, IGF-2, IL-1α, IL-4, IL-6, IL-10, IL-12β, IFNγ, PTPN3, PTPN15, PTPN22, TNF, RANTES. Genotypes of the insulin and IGF genes are also within the scope of the invention.

In the disclosure herein, including in the tables, the insulin gene and IGF genes are considered together. When the two genes are considered together (for example in the tables) then the IGF gene is IGF-1, which is located close to the insulin gene. However typing of genotypes in IGF-2 is also within the scope of the invention.

The invention concerns the detection of one or more genotypes. The genotype may be a SNP (single nucleotide polymorphism) or comprise more than one SNP (i.e. a haplotype), for example at least 2, 3, 4, 5, 6 or more SNPs may be typed (typically across a single gene or across different genes), and these SNPs are preferably the specific SNPs disclosed in Tables 1, 2, 3A or 3B. Thus in one embodiment 1, 2, 3, 4 or more of the SNPs shown in any of the haplotypes in Tables 3A or 3B are typed, so that all of the SNPs shown in the haplotypes in these tables do not have to be typed. However, of course, all of the SNPs in any of the haplotypes could be typed. In this context the term “type” refers to detecting the presence or absence of a genotype. Where more then one SNP is typed in an allele, at least 2, 3, 4 or more of the SNPs may be in linkage disequilibrium with each other and/or at least 2, 3, 4 or more of the SNPs may not be linkage disequilibrium with each other.

One or both alleles of any of the genes mentioned herein may be typed in the method. For the SNPs identified in Tables 1 and 2, the minor alleles were found to be associated with diabetes susceptibility (Table 1) or protection (Table 2).

The genotypes mentioned herein may be defined with reference to the flanking sequences or the primer sequences provided in the tables (Tables 5 to 9). Note that some of the tables show the reverse complement strands across the polymorphic position, but these can of course be used to unambiguously define the genotype (particularly in terms of its location in the gene). Representative sequences that flank the individual SNPs in Tables 1 and 2 are provided in Table 5. Representative sequences that flank the SNPs making up the haplotypes in Tables 3A and 3B are provided in Table 6. In both Tables 5 and 6 the SNPs are highlighted in bold. Table 6 provides a sequence map for the haplotypes in Tables 3A and 3B. Taking the SNPs from left to right in Tables 3A and 3B corresponds to the SNPs in bold going from top to bottom in Table 6. Determining a particular genotype may therefore involve determining the nucleotide present at the nucleotide position indicated in bold in the sequences in Tables 5 or 6. It will be understood that the exact sequences presented in Tables 5 and 6 will not necessarily be present in the dog to be tested. The sequence and thus the position of the SNP could for example vary because of deletions or additions of nucleotides in the genome of the dog.

The possession of the genotypes shown in Tables 1 and 3A indicates susceptibility to diabetes and the possession of the genotypes shown in Tables 2 and 3B indicates protection from diabetes. Thus the invention provides a method of identifying a dog which is susceptible or a dog which is protected from diabetes. Herein we describe the invention with respect to identifying a dog that is susceptible to diabetes, but it is understood that all embodiments disclosed in this context are also applicable to identifying a dog which is protected from diabetes.

In one embodiment a dog is deemed to be susceptible if it is found to possess a genotype shown in Table 1 or found to lack a genotype shown in Table 2, only if it is of the breed shown in the same line as the genotype, i.e. the method of the invention may be limited to detecting certain genotypes in certain breeds as defined in Table 1 and/or 2. In a further embodiment the method may be similarly limited to dogs which have one or more parents or grandparents from a breed as defined in Table 1 and/or 2, so that the method is carried out to detect the presence or absence of the genotype in a dog which has a parent or grandparent which is of the breed shown in the same line as the genotype in Table 1 and/or 2.

The detection of genotypes according to the invention may comprise contacting a polynucleotide of the dog with a specific binding agent for a genotype and determining whether the agent binds to the polynucleotide, wherein binding of the agent indicates the presence of the genotype, and lack of binding of the agent indicates the absence of the genotype.

The method is generally carried out in vitro on a sample from the dog, where the sample comprises nucleic acid (such as DNA) of the dog. The sample typically comprises a body fluid and/or cells of the individual and may, for example, be obtained using a swab, such as a mouth swab. The sample may be a blood, urine, saliva, skin, cheek cell or hair root sample. The sample is typically processed before the method is carried out, for example polynucleotide/DNA extraction may be carried out. The polynucleotide or protein in the sample may be cleaved either physically or chemically, for example using a suitable enzyme. In one embodiment the part of polynucleotide in the sample is copied or amplified, for example by cloning or using a PCR based method prior to detecting the genotype.

In the present invention, any one or more methods may comprise determining the presence or absence of one or more genotypes in the dog. The genotype is typically detected by directly determining the presence of the polymorphic sequence(s) in a polynucleotide of the dog. Such a polynucleotide is typically genomic DNA, mRNA or cDNA. The genotype may be detected by any suitable method such as those mentioned below.

A specific binding agent is an agent that binds with preferential or high affinity to the polynucleotide having the genotype, but does not bind or binds with only low affinity to other polynucleotides or polypeptides. The specific binding agent may be a probe or primer. The probe may be an oligonucleotide. The probe may be labelled or may be capable of being labelled indirectly. The binding of the probe to the polynucleotide or protein may be used to immobilise either the probe or the polynucleotide or protein.

Generally in the method, determination of the binding of the agent to the genotype can be carried out by determining the binding of the agent to the polynucleotide of the dog. However in one embodiment the agent is also able to bind the corresponding wild-type sequence, for example by binding the nucleotides which flank the genotype position, although the manner of binding to the wild-type sequence will be detectably different to the binding of a polynucleotide containing the genotype.

The method may be based on an oligonucleotide ligation assay in which two oligonucleotide probes are used. These probes bind to adjacent areas on the polynucleotide which contains the genotype, allowing after binding the two probes to be ligated together by an appropriate ligase enzyme. However the presence of single mismatch within one of the probes may disrupt binding and ligation. Thus ligated probes will only occur with a polynucleotide that contains the genotype, and therefore the detection of the ligated product may be used to determine the presence of the genotype.

In one embodiment the probe is used in a heteroduplex analysis based system. In such a system when the probe is bound to polynucleotide sequence containing the genotype it forms a heteroduplex at the site where the genotype occurs and hence does not form a double strand structure. Such a heteroduplex structure can be detected by the use of single or double strand specific enzyme. Typically the probe is an RNA probe, the heteroduplex region is cleaved using RNAase H and the genotype is detected by detecting the cleavage products.

The method may be based on fluorescent chemical cleavage mismatch analysis which is described for example in PCR Methods and Applications 3, 268-71 (1994) and Proc. Natl. Acad. Sci. 85, 4397-4401 (1998).

In one embodiment a PCR primer is used that primes a PCR reaction only if it binds a polynucleotide containing the genotype, for example a sequence- or allele-specific PCR system, and the presence of the genotype may be determined by the detecting the PCR product. Preferably the region of the primer which is complementary to the genotype is at or near the 3′ end of the primer. The presence of the genotype may be determined using a fluorescent dye and quenching agent-based PCR assay such as the Taqman PCR detection system.

The presence of the genotype may be determined based on the change which the presence of the genotype makes to the mobility of the polynucleotide or protein during gel electrophoresis. In the case of a polynucleotide single-stranded conformation genotype (SSCP) or denaturing gradient gel electrophoresis (DDGE) analysis may be used.

The presence of the polymorphism may be detected by means of fluorescence resonance energy transfer (FRET). In particular, the polymorphism may be detected by means of a dual hybridisation probe system. This method involves the use of two oligonucleotide probes that are located close to each other and that are complementary to an internal segment of a target polynucleotide of interest, where each of the two probes is labelled with a fluorophore. Any suitable fluorescent label or dye may be used as the fluorophore, such that the emission wavelength of the fluorophore on one probe (the donor) overlaps the excitation wavelength of the fluorophore on the second probe (the acceptor). A typical donor fluorophore is fluorescein (FAM), and typical acceptor fluorophores include Texas red; rhodamine, LC-640, LC-705 and cyanine 5 (Cy5).

In order for fluorescence resonance energy transfer to take place, the two fluorophores need to come into close proximity on hybridisation of both probes to the target. When the donor fluorophore is excited with an appropriate wavelength of light, the emission spectrum energy is transferred to the fluorophore on the acceptor probe resulting in its fluorescence. Therefore, detection of this wavelength of light, during excitation at the wavelength appropriate for the donor fluorophore, indicates hybridisation and close association of the fluorophores on the two probes. Each probe may be labelled with a fluorophore at one end such that the probe located upstream (5′) is labelled at its 3′ end, and the probe located downstream (3′) is labelled at is 5′ end. The gap between the two probes when bound to the target sequence may be from 1 to 20 nucleotides, preferably from 1 to 17 nucleotides, more preferably from 1 to 10 nucleotides, such as a gap of 1, 2, 4, 6, 8 or 10 nucleotides.

The first of the two probes may be designed to bind to a conserved sequence of the gene adjacent to a polymorphism and the second probe may be designed to bind to a region including one or more polymorphisms. Polymorphisms within the sequence of the gene targeted by the second probe can be detected by measuring the change in melting temperature caused by the resulting base mismatches. The extent of the change in the melting temperature will be dependent on the number and base types involved in the nucleotide polymorphisms.

Polymorphism typing may also be performed using a primer extension technique. In this technique, the target region surrounding the polymorphic site is copied or amplified for example using PCR. A single base sequencing reaction is then performed using a primer that anneals one base away from the polymorphic site (allele-specific nucleotide incorporation). The primer extension product is then detected to determine the nucleotide present at the polymorphic site. There are several ways in which the extension product can be detected. In one detection method for example, fluorescently labelled dideoxynucleotide terminators are used to stop the extension reaction at the polymorphic site. Alternatively, mass-modified dideoxynucleotide terminators are used and the primer extension products are detected using mass spectrometry. By specifically labelling one or more of the terminators, the sequence of the extended primer, and hence the nucleotide present at the polymorphic site can be deduced. More than one reaction product can be analysed per reaction and consequently the nucleotide present on both homologous chromosomes can be determined if more than one terminator is specifically labelled.

### Polynucleotides

The invention also provides a polynucleotide that comprises any genotype as disclosed herein. Thus the polynucleotide may comprise, or consist of, a fragment of the relevant gene which contains the polymorphism, and thus may comprise or be a fragment of any of the specific sequences disclosed herein. More particularly, the polynucleotide may comprise or be a fragment of any of the sequences in Tables 5 or 6.

The polynucleotide is typically at least 10, 15, 20, 30, 50, 100, 200 or 500 bases long, such as at least or up to 1 kb, 10 kb, 100 kb, 1000 kb or more in length. The polynucleotide will typically comprise flanking nucleotides on one or both sides of (5′ or 3′ to) the polymorphism; for example at least 2, 5, 10, 15 or more flanking nucleotides in total or on each side. Typically, the polynucleotide will be at least 70%, 80%, 90% or 95%, preferably at least 99%, even more preferably at least 99.9% identical to any of the specific sequences disclosed herein. Such numbers of substitutions and/or insertions and/or deletions and/or percentage identity may be taken over the entire length of the polynucleotide or over 50, 30, 15, 10 or less flanking nucleotides in total or on each side.

The polynucleotide may be RNA or DNA, including genomic DNA, synthetic DNA or cDNA. The polynucleotide may be single or double stranded. The polynucleotide may comprise synthetic or modified nucleotides, such as methylphosphonate and phosphorothioate backbones or the addition of acridine or polylysine chains at the 3′ and/or 5′ ends of the molecule.

A polynucleotide of the invention may be used as a primer, for example for PCR, or a probe. A polynucleotide of the invention may carry a revealing label. Suitable labels include radioisotopes such as ³²P or ³⁵S, fluorescent labels, enzyme labels or other protein labels such as biotin.

Polynucleotides of the invention may be used as a probe or primer which is capable of selectively binding to a genotype. The invention thus provides a probe or primer for use in a method according to the invention, which probe or primer is capable of selectively detecting the presence of a genotype. Preferably the probe is isolated or a recombinant nucleic acid. The probe may be immobilised on an array, such as a polynucleotide array.

Such primers, probes and other fragments will preferably be at least 10, preferably at least 15 or at least 20, for example at least 25, at least 30 or at least 40 nucleotides in length. They will typically be up to 40, 50, 60, 70, 100 or 150 nucleotides in length. Probes and fragments can be longer than 150 nucleotides in length, for example up to 200, 300, 400, 500, 600, 700 nucleotides in length, or even up to a few nucleotides, such as five or ten nucleotides, short of a full length polynucleotide sequence of the invention. Examples of primers and probes useful in the invention are provided in Tables 8 and 9. Polynucleotides of the invention may therefore comprise or consist of any of the sequences, or fragments of the sequences, provided in Tables 8 or 9, depending on which genotype is being typed.

The polynucleotides (e.g. primer and probes) of the invention may be present in an isolated or substantially purified form. They may be mixed with carriers or diluents which will not interfere with their intended use and still be regarded as substantially isolated. They may also be in a substantially purified form, in which case they will generally comprise at least 90%, e.g. at least 95%, 98% or 99%, of the polynucleotides or dry mass of the preparation.

### Homologues

Homologues of polynucleotide sequences are referred to herein. Such homologues typically have at least 70% homology, preferably at least 80, 90%, 95%, 97% or 99% homology, for example over a region of at least 15, 20, 30, 100 more contiguous nucleotides. The homology may be calculated on the basis of nucleotide identity (sometimes referred to as “hard homology”).

For example the UWGCG Package provides the BESTFIT program that can be used to calculate homology (for example used on its default settings) (Devereux et al (1984) Nucleic Acids Research 12, p387-395). The PILEUP and BLAST algorithms can be used to calculate homology or line up sequences (such as identifying equivalent or corresponding sequences (typically on their default settings), for example as described in Altschul S. F. (1993) J Mol Evol 36:290-300; Altschul, S, F et al (1990) J Mol Biol 215:403-10.

Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as default a word length (W) of 11, the BLOSUM62 scoring matrix (see Henikoff and Henikoff (1992) Proc. Natl. Acad. Sci. USA 89:10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two polynucleotide sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

### Linkage Disequilibrium

In the method of the invention the presence of a specific genotype can be inferred by typing a polymorphism which is in linkage disequilibrium with the specific genotype. Genotypes (SNPs or haplotypes) which are in linkage disequilibrium with each other in a population tend to be found together on the same chromosome. Typically one is found at least 30% of the times, for example at least 40%, 50%, 70% or 90%, of the time the other is found on a particular chromosome in individuals in the population. A polymorphism which is not a functional polymorphism, but is in linkage disequilibrium with a functional polymorphism, may act as a marker indicating the presence of the functional polymorphism. Genotypes which are in linkage disequilibrium with any of the genotypes mentioned herein are typically within 500 kb, preferably within 400 kb, 200 kb, 100 kb, 50 kb, 10 kb, 5 kb or 1 kb of the genotype.

### Detection Kit

The invention also provides a kit that comprises means for determining the presence or absence of one or more genotypes in a dog, such as any of the genotypes which can be typed to perform the method of the invention. In particular, such means may include a specific binding agent, probe, primer, pair or combination of primers, as defined herein which is capable of detecting or aiding detection of a genotype. The primer or pair or combination of primers may be sequence specific primers which only cause PCR amplification of a polynucleotide sequence comprising the genotype to be detected, as discussed herein. The kit may also comprise a specific binding agent, probe, primer, pair or combination of primers, which is capable of detecting the absence of the genotype. The kit may further comprise buffers or aqueous solutions.

The kit may additionally comprise one or more other reagents or instruments which enable any of the embodiments of the method mentioned above to be carried out. Such reagents or instruments may include one or more of the following: a means to detect the binding of the agent to the genotype, a detectable label such as a fluorescent label, an enzyme able to act on a polynucleotide, typically a polymerase, restriction enzyme, ligase, RNAse H or an enzyme which can attach a label to a polynucleotide, suitable buffer(s) or aqueous solutions for enzyme reagents, PCR primers which bind to regions flanking the genotype as discussed herein, a positive and/or negative control, a gel electrophoresis apparatus, a means to isolate DNA from sample, a means to obtain a sample from the individual, such as swab or an instrument comprising a needle, or a support comprising wells on which detection reactions can be carried out. The kit may be, or include, an array such as a polynucleotide array comprising the specific binding agent, preferably a probe, of the invention. The kit typically includes a set of instructions for using the kit.

### Screening for Therapeutic Agents

The present invention also relates to the use of the polymorphic polynucleotide sequence as a screening target for identifying therapeutic agents for the treatment of diabetes (i.e using a polynucleotide which comprises any of the genotypes disclosed herein). In one embodiment the invention provides a method for identifying an agent useful for the treatment of diabetes, which method comprises contacting the polynucleotide with a test agent and determining whether the agent is capable of modulating expression from the polynucleotide, for example of polypeptide.

The method may be carried out in vitro, either inside or outside a cell, or in vivo. In one embodiment the method is carried out on a cell, cell culture or cell extract.

The method may also be carried out in vivo in a non-human animal, for example which is transgenic for a genotype as defined herein. The transgenic non-human animal is typically of a species commonly used in biomedical research and is preferably a laboratory strain. Suitable animals include rodents, particularly a mouse, rat, guinea pig, ferret, gerbil or hamster. Most preferably the animal is a mouse.

Suitable candidate agents which may be tested in the above screening methods include antibody agents, for example monoclonal and polyclonal antibodies, single chain antibodies, chimeric antibodies and CDR-grafted antibodies. Furthermore, combinatorial libraries, defined chemical identities, peptide and peptide mimetics, oligonucleotides and natural agent libraries, such as display libraries may also be tested. The test agents may be chemical compounds, which are typically derived from synthesis around small molecules which may have any of the properties of the agent mentioned herein. Batches of the candidate agents may be used in an initial screen of, for example, ten substances per reaction, and the substances of batches which show modulation tested individually. The term ‘agent’ is intended to include a single substance and a combination of two, three or more substances. For example, the term agent may refer to a single peptide, a mixture of two or more peptides or a mixture of a peptide and a defined chemical entity. In one aspect of the invention, the test agent is a food ingredient, such as any of the type of food ingredients mentioned herein.

In one embodiment the therapeutic agent which is identified is used to treat a dog which comprises in its genome the same genotype that was present in the polynucleotide that was used for the screening.

### Treatment of Diabetes

The invention provides a method of treating a dog for diabetes. In one embodiment the method comprising identifying a dog which is susceptible to diabetes by a method of the invention, and administering to the dog an effective amount of a therapeutic agent which treats diabetes. The therapeutic agent may be any drug known in the art that may be used to treat diabetes, for example insulin, or may be an agent identified by a screening method as discussed previously.

The therapeutic agent may be administered in various manners such as orally, intracranially, intravenously, intramuscularly, intraperitoneally, intranasally, intrademally, and subcutaneously. The pharmaceutical compositions that contain the therapeutic agent will normally be formulated with an appropriate pharmaceutically acceptable carrier or diluent depending upon the particular mode of administration being used. For instance, parenteral formulations are usually injectable fluids that use pharmaceutically and physiologically acceptable fluids such as physiological saline, balanced salt solutions, or the like as a vehicle. Oral formulations, on the other hand, may be solids, for example tablets or capsules, or liquid solutions or suspensions.

The amount of therapeutic agent that is given to a dog will depend upon a variety of factors including the condition being treated, the nature of the dog under treatment and the severity of the condition under treatment. A typical daily dose is from about 0.1 to 50 mg per kg, preferably from about 0.1 mg/kg to 10 mg/kg of body weight, according to the activity of the specific inhibitor, the age, weight and conditions of the dog to be treated, the type and severity of the disease and the frequency and route of administration. Preferably, daily dosage levels are from 5 mg to 2 g.

### Customised Food

In one aspect, the invention relates to a customised diet for a dog that is susceptible to diabetes. In a preferred embodiment, the customised food is for a companion dog or pet, such as a dog. Such a food may be in the form of, for example, wet pet foods, semi-moist pet foods, dry pet foods and pet treats. Wet pet food generally has a moisture content above 65%. Semi-moist pet food typically has a moisture content between 20-65% and can include humectants and other ingredients to prevent microbial growth. Dry pet food, also called kibble, generally has a moisture content below 20% and its processing typically includes extruding, drying and/or baking in heat. The ingredients of a dry pet food generally include cereal, grains, meats, poultry, fats, vitamins and minerals. The ingredients are typically mixed and put through an extruder/cooker. The product is then typically shaped and dried, and after drying, flavours and fats may be coated or sprayed onto the dry product.

Accordingly, the present invention enables the preparation of customised food suitable for a dog which is susceptible to diabetes, wherein the customised dog food formulation comprises ingredients that prevent or alleviate diabetes, and/or does not comprise components that contribute to or aggravate diabetes. Such ingredients may be any of those known in the art to prevent or alleviate diabetes. Alternatively, screening methods as discussed herein may identify such ingredients. The customised dog food may be formulated to comprise a suitable level of simple carbohydrate (such as monosacharides and disaccharides). The preparation of customised dog food may be carried out by electronic means, for example by using a computer system.

In another embodiment, the customised food may be formulated to include functional or active ingredients that help prevent or alleviate diabetes.

The present invention also relates to a method of providing a customised dog food, comprising providing food suitable for an dog which is susceptible to diabetes to the dog, the dog's owner or the person responsible for feeding the dog, wherein the dog has been determined to be susceptible to diabetes by a method of the invention. In one aspect of the invention, the customised food is made to inventory and supplied from inventory, i.e. the customised food is pre-manufactured rather than being made to order. Therefore according this aspect of the invention the customised food is not specifically designed for one particular dog but instead is suitable for more than one dog. For example, the customised food may be suitable for any dog that is susceptible to diabetes. Alternatively, the customised food may be suitable for a sub-group of dogs that are susceptible to diabetes, such as dogs of a particular breed, size or lifestage. In another embodiment, the food may be customised to meet the nutritional requirements of an individual dog.

### Bioinformatics

The sequences of the genotypes may be stored in an electronic format, for example in a computer database. Accordingly, the invention provides a database comprising information relating to genotype sequences. The database may include further information about the genotype, for example the level of association of the genotype with diabetes or the frequency of the genotype in the population. In one aspect of the invention, the database further comprises information regarding the food components which are suitable and the food components which are not suitable for dogs who possess a particular genotype.

A database as described herein may be used to determine the susceptibility of a dog to diabetes. Such a determination may be carried out by electronic means, for example by using a computer system (such as a PC). Typically, the determination will be carried out by inputting genetic data from the dog to a computer system; comparing the genetic data to a database comprising information relating to genotypes; and on the basis of this comparison, determining the susceptibility of the dog to diabetes.

The invention also provides a computer program comprising program code means for performing all the steps of a method of the invention when said program is run on a computer. Also provided is a computer program product comprising program code means stored on a computer readable medium for performing a method of the invention when said program is run on a computer. A computer program product comprising program code means on a carrier wave that, when executed on a computer system, instruct the computer system to perform a method of the invention is additionally provided.

The invention also provides an apparatus arranged to perform a method according to the invention. The apparatus typically comprises a computer system, such as a PC. In one embodiment, the computer system comprises: means 20 for receiving genetic data from the dog; a module 30 for comparing the data with a database 10 comprising information relating to genotypes; and means 40 for determining on the basis of said comparison the susceptibility of the dog to diabetes.

### Food Manufacturing

In one embodiment of the invention, the manufacture of a customised dog food may be controlled electronically. Typically, information relating to the genotype present in a dog may be processed electronically to generate a customised dog food formulation. The customised dog food formulation may then be used to generate electronic manufacturing instructions to control the operation of food manufacturing apparatus. The apparatus used to carry out these steps will typically comprise a computer system, such as a PC, which comprises means 50 for processing the nutritional information to generate a customised dog food formulation; means 60 for generating electronic manufacturing instructions to control the operation of food manufacturing apparatus; and a food product manufacturing apparatus 70.

The food product manufacturing apparatus used in the present invention typically comprises one or more of the following components: container for dry pet food ingredients; container for liquids; mixer; former and/or extruder; cut-off device; cooking means (e.g. oven); cooler; packaging means; and labelling means. A dry ingredient container typically has an opening at the bottom. This opening may be covered by a volume-regulating element, such as a rotary lock. The volume-regulating element may be opened and closed according to the electronic manufacturing instructions to regulate the addition of dry ingredients to the pet food.

Dry ingredients typically used in the manufacture of pet food include corn, wheat, meat and/or poultry meal. Liquid ingredients typically used in the manufacture of pet food include fat, tallow and water. A liquid container may contain a pump that can be controlled, for example by the electronic manufacturing instructions, to add a measured amount of liquid to the pet food.

In one embodiment, the dry ingredient container(s) and the liquid container(s) are coupled to a mixer and deliver the specified amounts of dry ingredients and liquids to the mixer. The mixer may be controlled by the electronic manufacturing instructions. For example, the duration or speed of mixing may be controlled. The mixed ingredients are typically then delivered to a former or extruder. The former/extruder may be any former or extruder known in the art that can be used to shape the mixed ingredients into the required shape. Typically, the mixed ingredients are forced through a restricted opening under pressure to form a continuous strand. As the strand is extruded, it may be cut into pieces (kibbles) by a cut-off device, such as a knife. The kibbles are typically cooked, for example in an oven. The cooking time and temperature may be controlled by the electronic manufacturing instructions. The cooking time may be altered in order to produce the desired moisture content for the food. The cooked kibbles may then be transferred to a cooler, for example a chamber containing one or more fans.

The food manufacturing apparatus may comprise a packaging apparatus. The packaging apparatus typically packages the food into a container such as a plastic or paper bag or box. The apparatus may also comprise means for labelling the food, typically after the food has been packaged. The label may provide information such as: ingredient list; nutritional information; date of manufacture; best before date; weight; and species and/or breed(s) for which the food is suitable.

### Breeding Tool

In order to avoid the problems of diseases associated with inbreeding, it would be advantageous to select dogs within a breed for breeding that are not genetically predisposed to certain diseases such as diabetes. Accordingly, the invention provides a method of selecting a dog which is not susceptible to diabetes, the method comprising determining whether the dog is susceptible to diabetes using the method of the invention and optionally breeding the selected dog. More specifically, the invention provides a method of selecting one or more dogs for breeding with a subject dog, the method comprising:

(a) determining the susceptibility to diabetes of the subject dog and of each dog in a test group of two or more dogs of the same breed and of the opposite sex to the subject dog; and

(b) selecting one or more dogs from the test group for breeding with the subject dog, wherein the selected dog is not susceptible to diabetes.

The invention is illustrated by the following Examples:

### EXAMPLES

We genotyped a canine diabetic cohort (n=489), comprising 20 pedigree breeds and crossbreeds, for single nucleotide polymorphisms (SNPs) in candidate genes. Cases were compared to breed-matched controls selected from a control dataset of 1000 dogs. Control populations were checked for Hardy-Weinberg compliance. Allele frequencies were compared between controls and cases using χ², and haplotype analysis using an association score test.

### Methods and Materials

CTLA4, Rantes, IFNg, IGF, Insulin and some TNF SNPs were analysed by Taqman the others were analysed by Sequenom. Sequenom is a simple, robust method of accurately genotyping multiple SNPs in a single reaction. It uses matrix-assisted laser desorption/ionisation time-of-flight mass spectrometry (MALDI-TOF MS). The assay is based on probes annealing adjacent to the SNP. DNA polymerase and terminator nucleotides extend the primer through the polymorphic site, generating allele-specific extension products, each with a unique molecular mass. These masses are analysed by MALDI-TOF MS, and genotypes assigned on the basis of mass. Primers and probes were designed using Assay Design software Version 3, and synthesised by Metabion (Germany). The Taqman primer and probe sequences used are provided in Table 8. The Sequenom primers are provided in Table 9.

Primers were diluted to 100 μM and plexes pooled to contain 500 nm of each forward and reverse primer. Probes were diluted to 400 μM and probe pools were split into 50% high mass and 50% low mass probes. Probe pools contained 26 μl of each low mass probe and 52 μl of each high mass probe in a final volume of 1.5 ml.

For each PCR reaction, 15 ng DNA was plated into a 384 well plate, and dried down at room temperature overnight. PCR was carried out in a 5 μl volume on a PTC-225 MJ Tetrad cycler (384 well). Each reaction contained 1.25× HotStarTaq PCR buffer, 1.625 mM MgCl₂, 500 μM of each dNTP, 0.5 U of HotStarTaq and 100 nm primer pool and was amplified as follows: 95° C. for 15 minutes; 35 cycles of 95° C. for 20 seconds, 56° C. for 30 seconds, 72° C. for 1 minute; 72° C. for 3 minutes. The reaction was then kept at 4° C.

Following PCR, the reactions were treated with 0.3 U shrimp alkaline phosphatase (SAP) to inactivate any dNTPs leftover from the reaction. Reactions were incubated at 37° C. for 20 minutes, and denatured at 80° C. for 5 minutes. iPLEX primer extension was carried out on a dyad PCR engine. Reactions contained 0.22× iPLEX buffer, 1× iLPEX termination mix, 0.625 μm low mass primer, 1.25 μm high mass primer and 1× iPLEX enzyme, and were amplified as follows: 94° C. for 30 seconds, 40 cycles of 94° C. for 5 seconds, 5 cycles of 52° C. for 5 seconds, 80° C. for 5 seconds, and a final extension of 72° C. for 3 minutes. Samples were diluted with 25 μl water, and desalted using 6 mg resin before being centrifuged for 5 minutes at 4,000 rpm in a Jouan CR4 centrifuge, and spotted onto a SpectroCHIP using a Sequenom mass array nanodispenser (Samsung).

### Statistics

Minor allele frequencies were compared between cases and controls using the BCgene ‘fast association’ analysis tool. Chi-squared, p values, odds ratios (OR) and confidence intervals (CI) were calculated for each SNP by breed. Data were taken for further analysis if the chi-squared was greater than 3.84, the p value less than 0.05 and the control population was in HWE. SNPs in which the diabetic populations were not in HWE were included in the analysis as this could be a consequence of the disease. The significance of these data was checked using the programme CLUMP (Sham and Curtis, 1995). CLUMP uses the Monte Carlo approach to generate chi-squared and p values in a 2×n contingency table. Repeated simulations of the data are carried out (1000 for this study), and the frequency of chi-squared values in the simulated data which are associated with the observed data are counted, giving unbiased significance levels. CLUMP generates four chi-squared statistics (T1-T4), for the purpose of this study the normal chi-squared statistic (T1) was used. This resulted in a final set of significant SNPs with OR and CI greater than 1 (susceptibility alleles; Table 1) or with OR and CI less than 1 (protective alleles; Table 2). A sequence map of these susceptibility and protective SNPs is set out in Table 5. The minor allele for each SNP in Table 1 is the susceptibility allele. Likewise, the minor allele for each SNP in Table 2 is the protective allele. The location of each SNP with reference to flanking sequence is represented in bold in each sequence in Table 5.

Haplotypes for each gene were estimated from the data-set using Helix Tree version 4.10 (www.goldenhelix.com).

Since there is considerable inter-breed variability in observed gene locus haplotype frequencies, further analysis was performed by stratifying breeds according to their diabetes risk status, in an attempt to determine whether haplotypes shared by different breeds would segregate with the different risk groups. The breed profile of the diabetic dog population illustrates marked differences in diabetes risk from the Samoyed (with an odds ratio of 17.3) to the Boxer (with an odds ratio of 0.07). This range of diabetes risk across breeds is reminiscent of what is seen in different human populations where disease prevalence can be extremely high in some ethnic groups originating from a limited gene pool. In particular, diabetes and other autoimmune conditions are very prevalent in a number of discrete human populations such as indigenous North Americans, where there are exceptionally raised frequencies of high risk alleles and haplotypes.

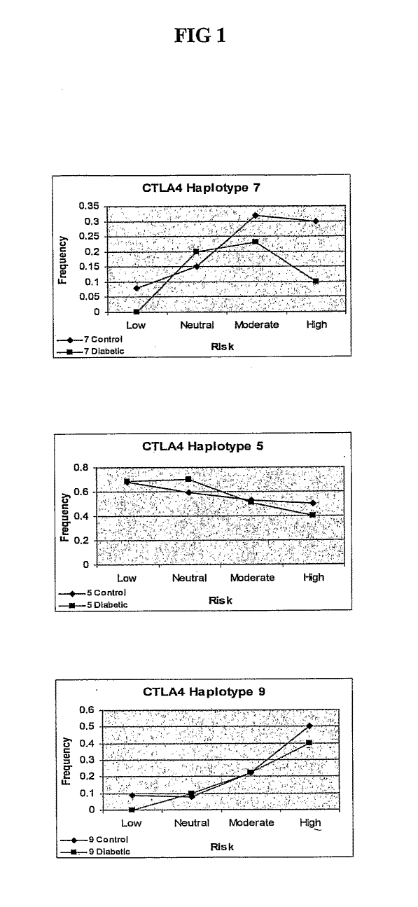

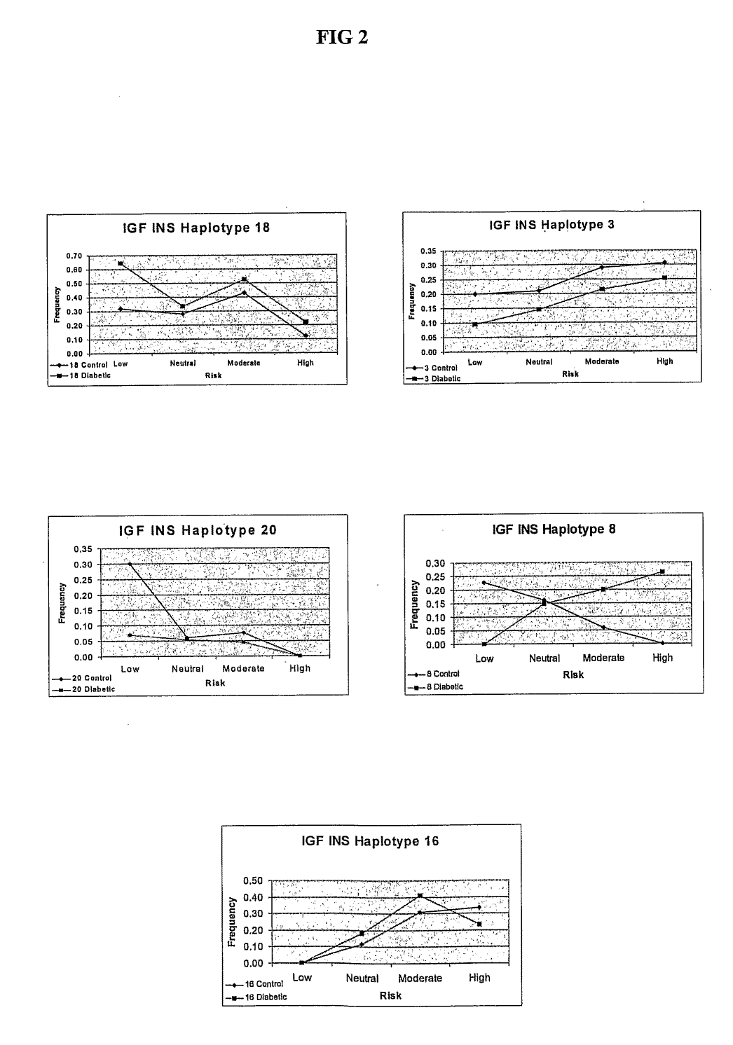

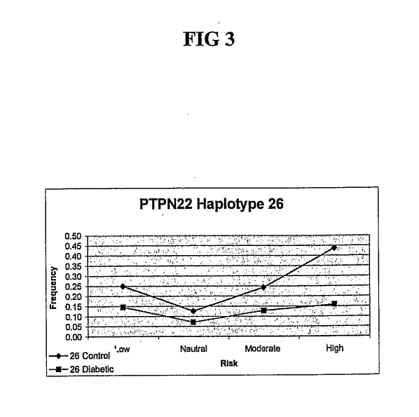

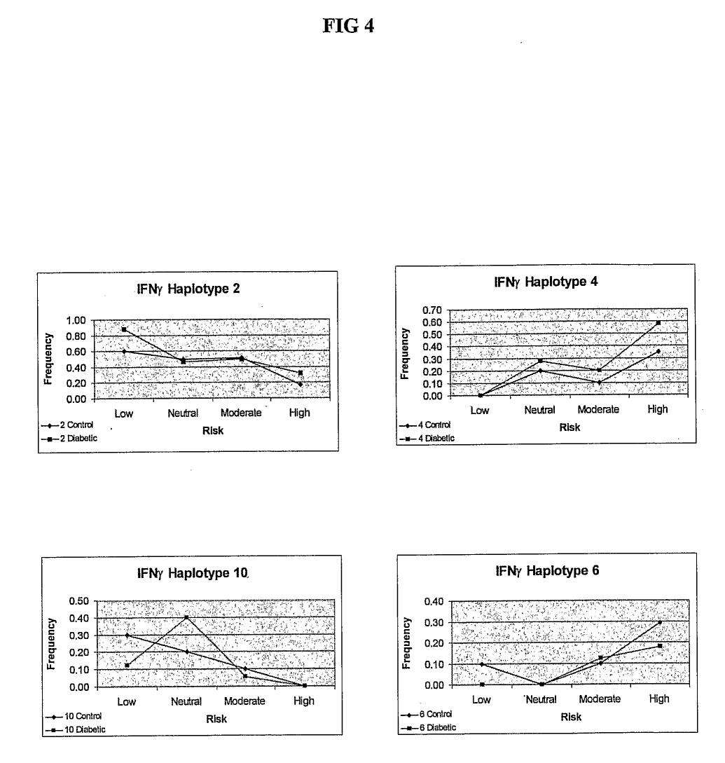

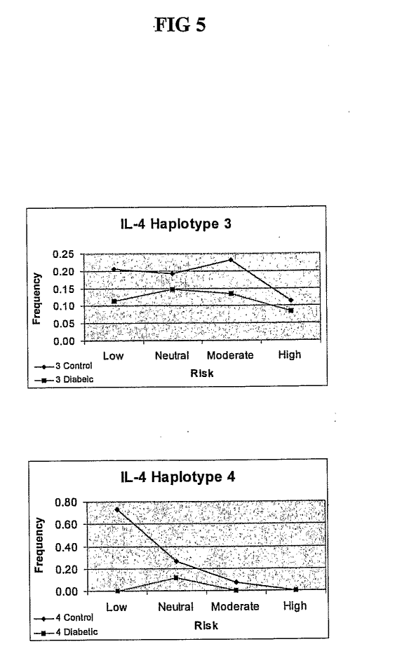

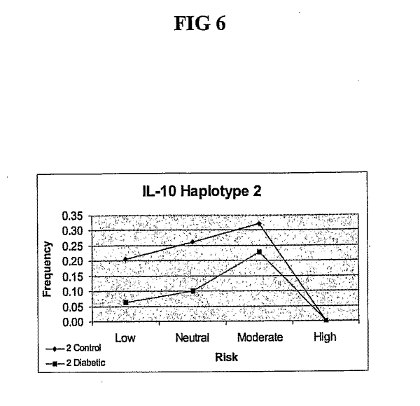

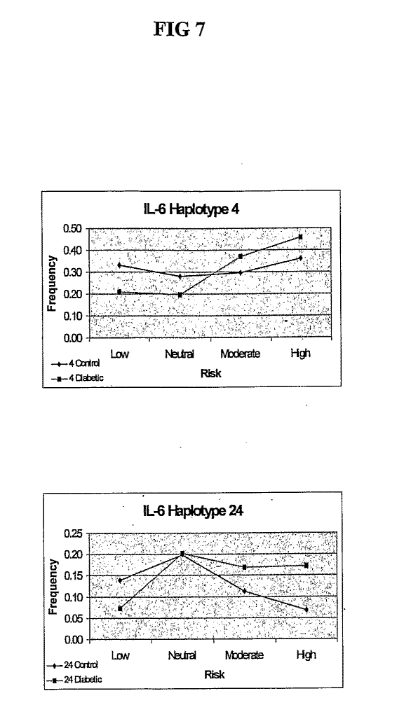

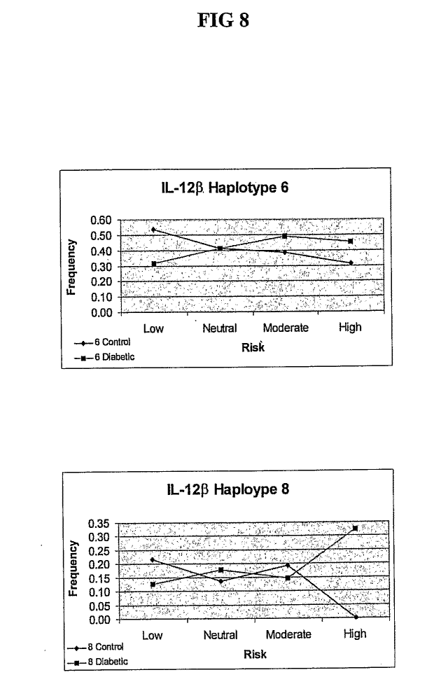

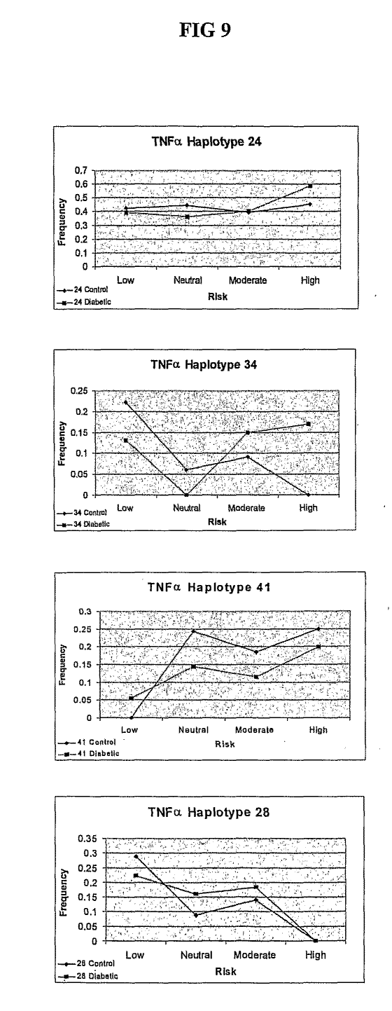

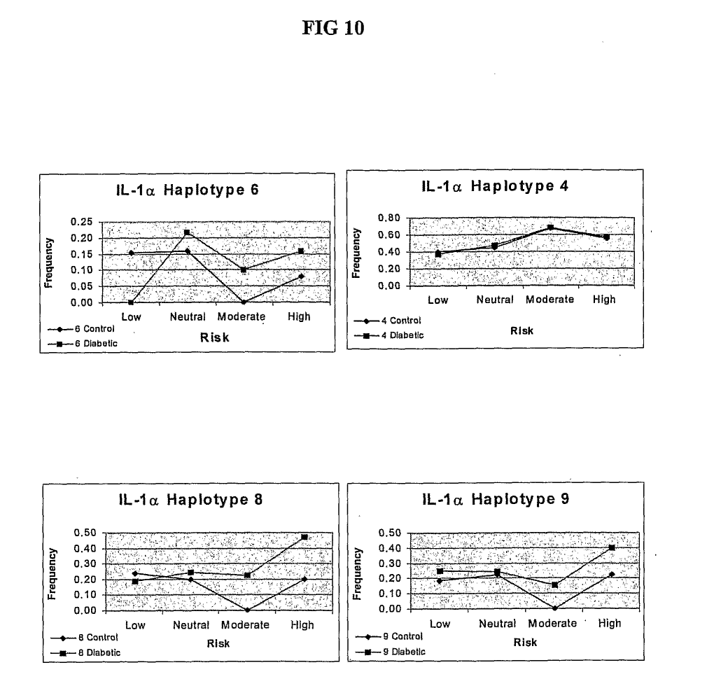

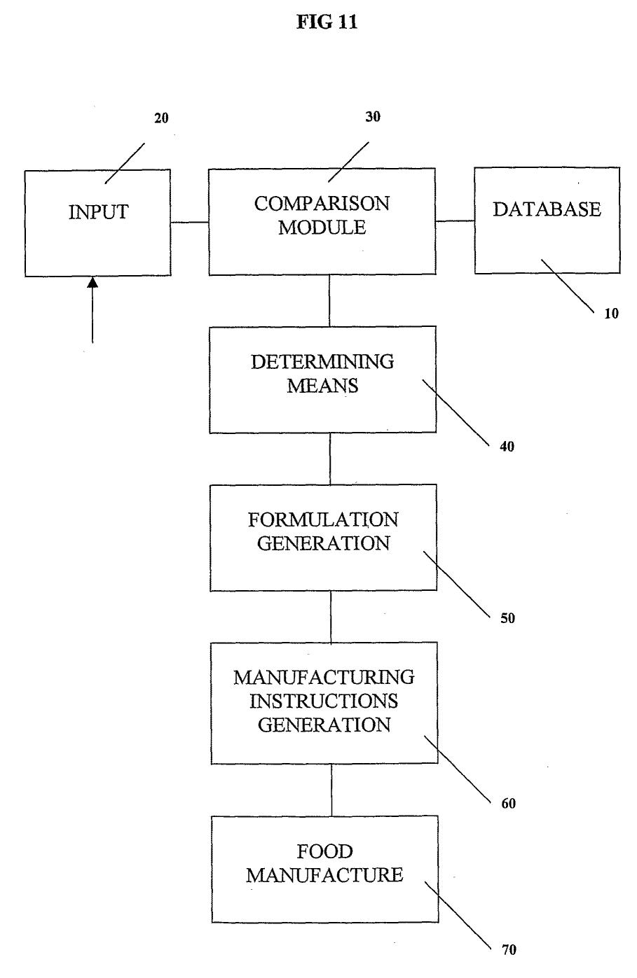

To minimise breed-specific bias in the analysis, we chose to analyse the data in groups of breeds stratified into diabetes risk groups ranging from high risk (e.g Samoyeds, Tibetan terriers, and Cain terriers) through to breeds exhibiting clear protection (e.g Boxers, German shepherd dogs and Golden Retrievers—see Table 4 and FIGS. 1 to 10).

The frequency of dogs carrying the suspected susceptibility haplotypes and protective haplotypes was examined for cases and controls in each risk group to determine whether the haplotype was generally observed more frequently in cases than controls, particularly in the high risk breeds (see haplotype frequency graphs for individual candidate haplotypes in FIGS. 1 to 10). When stratified in this way two observations could be made. Firstly, the frequency of the susceptible haplotypes were generally higher in those breeds assigned to the higher risk categories. Secondly, the reverse was generally observed for the protective haplotypes.

Tables 3A and 3B show susceptible and protective haplotypes deduced from the shape of the graph and distribution across high, low and neutral risk breeds (FIGS. 1 to 10). For a haplotype to be classed as protective, the frequency of that haplotype decreases as risk category increases and the reverse is true for a susceptibility haplotype, i.e. haplotype frequency increases as risk category increases. The SNPs constituting the haplotypes in Tables 3A and 3B are mapped out with reference to flanking sequence in Table 6. The SNPs are highlighted in bold in the sequences in Table 6. Taking the SNPs from left to right in the haplotypes in Table 3 corresponds to the SNPs in bold going from top to bottom in Table 6.

TABLE 1
Susceptibility Alleles.
The minor allele is the susceptibility allele.
OR and CI greater than 1

| SNP | | | | | | | | | | | |
|---|---|---|---|---|---|---|---|---|---|---|---|
| ref/ | | | | | | | | | | | |
| SEQ | | | | | | | | | | | |
| ID | | | | | | | CI | CI | Minor | Case | Control |
| NO: | Breed | SNP | X2 | p | T1p | OR | min | max | allele | (n) | (n) |
| 3 | Collie | IL-4 25Y336 | 8.37 | 0.004 | 0.004 | 15.85 | 2.40 | 106.00 | C | 14 | 20 |
| 10 | Dachshund | IL-12b 02M407 | 5.18 | 0.023 | 0.030 | 3.20 | 1.16 | 8.85 | A | 26 | 42 |
| 11 | Dachshund | IL-12b 03R196 | 4.48 | 0.034 | 0.042 | 2.97 | 1.07 | 8.26 | G | 26 | 40 |
| 33 | Labrador | CTLA4 11Y540 | 4.97 | 0.026 | 0.043 | 3.71 | 1.09 | 12.63 | T | 104 | 182 |
| 21 | Poodle | PTPN 3 | 6.15 | 0.013 | 0.017 | 5.23 | 1.34 | 20.45 | G | 14 | 34 |
| 33 | Samoyed | CTLA4 11Y540 | 7.39 | 0.007 | 0.004 | 12.54 | 3.25 | 48.33 | T | 28 | 16 |
| 4 | Schnauzer | IL-4 1K110 | 8.18 | 0.004 | 0.005 | 14.38 | 1.64 | 126.08 | T | 12 | 30 |
| 5 | Schnauzer | IL-4 2M351 | 6.06 | 0.014 | 0.025 | 6.80 | 1.31 | 35.41 | C | 14 | 32 |
| 13 | Cavalier King Charles | IL-10 11R124 | 5.17 | 0.023 | 0.040 | 3.30 | 1.16 | 9.38 | A | 34 | 28 |
| | Spaniel | | | | | | | | | | |
| 14 | Cavalier King Charles | IL-10 13Y85 | 7.07 | 0.008 | 0.013 | 3.85 | 1.40 | 10.59 | T | 38 | 30 |
| | Spaniel | | | | | | | | | | |
| 15 | Cavalier King Charles | IL-10 14R553 | 5.37 | 0.020 | 0.038 | 4.05 | 1.21 | 13.54 | G | 24 | 24 |
| | Spaniel | | | | | | | | | | |
| 16 | Cavalier King Charles | IL-10 1R105 | 5.78 | 0.016 | 0.026 | 3.33 | 1.23 | 9.03 | A | 36 | 32 |
| | Spaniel | | | | | | | | | | |
| 17 | Cavalier King Charles | IL-10 1R117 | 5.17 | 0.023 | 0.040 | 3.30 | 1.16 | 9.38 | A | 34 | 28 |
| | Spaniel | | | | | | | | | | |
| 18 | Cavalier King Charles | IL-10 1R218 | 5.17 | 0.023 | 0.040 | 3.30 | 1.16 | 9.38 | G | 34 | 28 |
| | Spaniel | | | | | | | | | | |
| 19 | Cavalier King Charles | IL-10 2R420 | 6.29 | 0.012 | 0.024 | 3.76 | 1.31 | 10.81 | G | 34 | 28 |
| | Spaniel | | | | | | | | | | |
| 20 | Cavalier King Charles | IL-10 6Y135 | 7.28 | 0.007 | 0.013 | 4.00 | 1.43 | 11.18 | C | 36 | 30 |
| | Spaniel | | | | | | | | | | |
| 8 | Cocker Spaniel | IL-6 20R191 | 14.70 | 0.000 | 0.001 | 8.72 | 2.68 | 28.35 | G | 26 | 40 |
| 6 | Cocker Spaniel | IL-6 6R431 | 4.84 | 0.028 | 0.040 | 2.81 | 1.10 | 7.17 | G | 34 | 50 |
| 28 | Cocker Spaniel | TNF 10513 | 6.94 | 0.008 | 0.016 | 4.01 | 1.38 | 11.66 | A | 32 | 48 |
| 35 | Border Terrier | CTLA4 12K291 | 7.57 | 0.006 | 0.012 | 13.27 | 2.06 | 85.64 | G | 18 | 24 |
| 12 | Border Terrier | IL-12b 01Y90 | 5.17 | 0.023 | 0.023 | 9.62 | 1.01 | 91.16 | C | 18 | 26 |
| 36 | Jack Russell Terrier | IFNg 5M532 | 4.44 | 0.035 | 0.036 | 2.54 | 1.05 | 6.11 | C | 34 | 74 |
| 23 | Jack Russell Terrier | INS 8 | 7.87 | 0.005 | 0.012 | 4.31 | 1.48 | 12.52 | G | 28 | 70 |
| 7 | West Highland White | IL-6 6K372 | 7.96 | 0.005 | 0.010 | 7.07 | 1.52 | 32.94 | T | 68 | 68 |
| | Terrier | | | | | | | | | | |
| 28 | West Highland White | TNF 10513 | 6.46 | 0.011 | 0.010 | 6.15 | 1.29 | 29.33 | A | 62 | 66 |
| | Terrier | | | | | | | | | | |
| 8 | Yorkshire Terrier | IL-6 20R191 | 7.09 | 0.008 | 0.013 | 3.97 | 1.38 | 11.39 | G | 44 | 52 |
| 9 | Yorkshire Terrier | IL-6 20R240 | 5.67 | 0.017 | 0.029 | 2.85 | 1.18 | 6.91 | A | 56 | 88 |

TABLE 2
Protective alleles.
The minor allele is the protective allele.
OR and CI less than 1

| SNP | | | | | | | | | | | |
|---|---|---|---|---|---|---|---|---|---|---|---|
| ref/ | | | | | | | | | | | |
| SEQ | | | | | | | | | | | |
| ID | | | | | | | | CI | Minor | Case | Control |
| NO: | Breed | SNP | X2 | p | T1p | OR | CI min | max | allele | (n) | (n) |
| 1 | Collie | IL-4 13S97 | 5.79 | 0.016 | 0.039 | 0.14 | 0.03 | 0.78 | C | 16 | 22 |
| 2 | Collie | IL-4 8R458 | 5.63 | 0.018 | 0.029 | 0.14 | 0.03 | 0.80 | G | 16 | 20 |
| 31 | Crossbreed | CTLA411R3 | 4.27 | 0.039 | 0.039 | 0.40 | 0.16 | 0.98 | G | 180 | 66 |
| | | 86 | | | | | | | | | |
| 32 | Crossbreed | CTLA4 | 6.75 | 0.009 | 0.016 | 0.33 | 0.14 | 0.79 | T | 178 | 72 |
| | | 11Y437 | | | | | | | | | |
| 22 | Crossbreed | PTPN 15 | 5.66 | 0.017 | 0.031 | 0.21 | 0.05 | 0.85 | T | 162 | 72 |
| 6 | Dachshund | IL-6 6R431 | 19.24 | 0.000 | 0.001 | 0.06 | 0.03 | 0.16 | G | 28 | 48 |
| 23 | Labrador | INS 8 | 4.79 | 0.028 | 0.019 | 0.05 | 0.22 | 0.93 | G | 96 | 156 |
| 33 | Schnauzer | CTLA4 | 4.76 | 0.029 | 0.030 | 0.16 | 0.03 | 0.09 | T | 16 | 28 |
| | | 11Y540 | | | | | | | | | |
| 24 | Cocker Spaniel | INS1 | 6.73 | 0.009 | 0.020 | 0.11 | 0.03 | 0.37 | C | 28 | 58 |
| 25 | Border Terrier | IGF2 10 | 8.58 | 0.003 | 0.007 | 0.11 | 0.03 | 0.49 | A | 16 | 22 |
| 7 | Border Terrier | IL-6 6K372 | 4.99 | 0.025 | 0.031 | 0.21 | 0.05 | 0.88 | T | 20 | 26 |
| 1 | Cairn Terrier | IL-4 13S97 | 7.18 | 0.007 | 0.018 | 0.06 | 0.007 | 0.48 | C | 26 | 16 |
| 2 | Cairn Terrier | IL-4 8R458 | 8.83 | 0.003 | 0.015 | 0.06 | 0.01 | 0.56 | G | 26 | 12 |
| 26 | Cairn Terrier | TNF 9585 | 8.15 | 0.004 | 0.009 | 0.068 | 0.01 | 0.44 | C | 26 | 18 |
| 7 | Jack Russell | IL-6 6K372 | 4.61 | 0.032 | 0.035 | 0.39 | 0.16 | 0.93 | T | 36 | 78 |
| | Terrier | | | | | | | | | | |
| 29 | West Highland | CTLA4 | 5.49 | 0.019 | 0.023 | 0.23 | 0.06 | 0.86 | A | 66 | 70 |
| | White Terrier | 11R124 | | | | | | | | | |
| 30 | West Highland | CTLA4 | 4.61 | 0.032 | 0.045 | 0.25 | 0.07 | 0.96 | A | 72 | 68 |
| | White Terrier | 11R204 | | | | | | | | | |
| 31 | West Highland | CTLA4 | 4.61 | 0.032 | 0.045 | 0.25 | 0.07 | 0.96 | G | 72 | 68 |
| | White Terrier | 11R386 | | | | | | | | | |
| 34 | West Highland | CTLA4 | 5.85 | 0.016 | 0.030 | 0.18 | 0.04 | 0.84 | C | 68 | 68 |
| | White Terrier | 12Y232 | | | | | | | | | |
| 27 | West Highland | TNF 9367 | 5.77 | 0.016 | 0.026 | 0.35 | 0.15 | 0.84 | C | 64 | 66 |
| | White Terrier | | | | | | | | | | |

TABLE 3A
Susceptible haplotypes

CTLA4, ID 7 - GGGCAGACCCTTGGC
CTLA4, ID 9 - GGGCAGACTATTTGC
IGF INS, ID 3 - AACAGACAAAT
IGF INS, ID 8 - GGAGAGCAGGC
IGF INS, ID 16 - GGCAAGTGGGC
PTPN22, ID 26 - GAGCAGGGGGA
IFNg, ID 4 - AACCT
IFNg, ID 6 - ACACT
IL6, ID 4 - GACGGATGAGG
IL12b, ID 6 - TACCTCTAGGT
TNFa, ID 24 - AAAGGTCTAATTATTGC
IL12b, ID 8 - TACTACCAAGT
TNFa, ID 34 - AAAGGAGTAATAATTGC
TNFa, ID 41 - AAAGATCACATTCTTGC
IL-1a, ID 4 - GACTTG
IL-1a, ID 8 - TACCTG
IL-1a, ID 9 - TACTTA
IL-1a, ID 6 - GCCTTG
IL6, ID 24 - TACAGATGAGG

TABLE 3B
Protective haplotypes

CTLA4, ID 5 - GGGCAGACCATTTGC
IGF INS, ID18 - GGCAGACAAAT
IGF INS, ID 20 - GGCAGACAGGC
IFNg, ID 10 - GAACT
IL4, ID 4 - TCGAACAG
IL10, ID 2 - CAGAGTAACCAGGA
TNFa, ID 28 - AAAGGTCACATTCTTGC
IL4, ID 3 - TCCAGGAG
IFNg, ID 2 - AAACT

TABLE 4
Segregation of breeds into different risk groups.

| Risk | | |
|---|---|---|
| group | OR | Breed |
| high | 17.30 | Samoyed |
| high | 6.93 | Tibetan Terrier |
| high | 6.77 | Cairn Terrier |
| moderate | 3.60 | Bichon Frise |
| moderate | 3.48 | Yorkshire Terrier |
| moderate | 3.18 | Miniature Schnauzer |
| moderate | 2.89 | Border Collie |
| moderate | 2.83 | Dachshund |
| moderate | 2.51 | Border Terrier |
| moderate | 2.40 | Miniature Poodle |
| neutral | 1.74 | Rottweiler |
| neutral | 1.70 | WHW terrier |
| neutral | 1.48 | Jack Russell Terrier |
| neutral | 1.45 | CKC Spaniel |
| neutral | 1.22 | Dobermann |
| neutral | 0.97 | Labrador |
| neutral | 0.78 | Crossbreed |
| neutral | 0.75 | Cocker Spaniel |
| protected | 0.19 | Golden Retriever |
| protected | 0.15 | German Shepherd dog |
| protected | 0.07 | Boxer |

TABLE 5
Sequence Map of SNPs

| | | |
|---|---|---|
| | SNP ref/ | |
| | SEQ ID | |
| SNP | NO: | Sequence |

| | | |
|---|---|---|
| IL4 13S97 | 1 | GCTAGGCGTGAGATCAGAGGAAGCTTCTGGAAGAGGSTGCAGTTGAGCTGGGCCATGGACACAA |
| IL4 8R458 | 2 | TCAAACTTAGTATTGATAAATTGAACTCCTGATCTTCTGCTCAACCTCCARCACTGCTCTGCGCTCAATTTTC |
| | | TGGGCACCAGCCCTCTCCCAAAAGGCT |
| IL4 25Y336 | 3 | CCTTTGGGTATATTTCCAGAAGTAGAATTACTGGATCATGTAGCATTTGTATTTTYAGTTTTTTGAGGATTTT |
| | | TCATACTGTTTTCCATAGTGGCTGCACCAG |
| IL41K110 | 4 | TGATTTGCCACTTCTGGATGTTTCATATAAATGGAATCATGTAGCCTTTTGTATCTGKCTTCTTTCACTTACC |
| | | CTAGTGTTTCTAAGGTTCATCCATATTGTAGCG |
| IL4 2M351 | 5 | AACCTTGGATATTGTGTGTTAATTTCTGTATTGAAAAGTGAGGGTTCACTTCATTTGTACTACCCCTTCCAMA |
| | | TTTTTTATAGTGAATTTATTTTCAGATCTTGTATTACC |
| IL6 6R431 | 6 | ATATGAGAAAAAGCAATCCCACACTACAGAGGCTTTTTGCAAGCATCACAGTGGRGCTGGGAGAGGTGGCTTC |
| | | ATTCAGCGCAGGAGAGAGGACTCGGCTGGCAGTGTC |
| IL6 6K372 | 7 | AGCTAAACCACTAAGCCACCAGGGCTGCCCCCAAGTCATATTTTCTAAAACATAKATATATATGAGAAAAAGC |
| | | AATCCCACACTACAGAGGCTTTTTG |
| IL6 20R191 | 8 | TCAATCCCAGCCCCTGTACACACTTTTATGGACRTAGGAGAAGGGACTTCCCAAAGTCACCCAGCTAGAAGG |
| IL6 20R240 | 9 | GGGACTTCCCAAAGTCACCCAGCTAGAAGGTAAGGCACAGRCCCAGATTTTAAATCCAGGTCTAATTGCCTCC |
| | | GGGCGTCCTACTCTTAAC |
| IL12b 02M407 | 10 | GGGTATATCAATATTTTAGGGTCTTCTCCCAAAGAACCTCTTGATTTTCAGMGCTTATGGGCTTGAACATGGG |
| | | TTAAACCAGTGGTTCTCAAAGTGTGGTCT |
| IL12b 03R196 | 11 | AAACAAGGAGAGAAACTAAACCTGGCCACCAGATCATTGCCRTAATTTGAAATCACCTCTAATTGTCTCCCAC |
| | | CACCACCA |
| IL12b 01Y90 | 12 | TTTCCCTACAGCCAGGCACGACTTTTTACCCTACYATTGTACACAAAACAGACATATC |
| IL10 11R124 | 13 | CACTCGCTAGCCACGCTTTTTAGGCCAACCCCGCRTCGCCTCTCCCAAGGCGACTGGGTG |
| IL10 13Y85 | 14 | ACAGACGCCATAGTCTTCCTATAAACTCAGTYCTTTAAGACATTATCCTTAAACTCTAAAAGATCATGCTG |
| IL10 14R553 | 15 | GTCACAGTTTACTGAGCACTTATTTTGAGCCAGCCRGTGCTAGTTCTGTACATGTCAGCCATAGGGTAT |
| IL10 1R105 | 16 | GCTCTTCCTAGTTACTGTCTTCACTGGGGAGGTAR(105)GAAAAGCTCCTR(117)TAGAAGGAGAAGGTCA |
| | | AGGTACATCAAGGGACCC |
| IL10 1R117 | 17 | GCTCTTCCTAGTTACTGTCTTCACTGGGGAGGTAR(105)GAAAAGCTCCTR(117)TAGAAGGAGAAGGTCA |
| | | AGGTACATCAAGGGACCC |
| IL10 1R218 | 18 | CCGCCCTCTCCTTTCCTTATTAGAGGTARAGCAACTTTCCTCACTGCACCTGCCTACCGCCCCTGC |
| IL10 2R420 | 19 | ACTTGGGGAAACTGAGGCTCTTCCCAGTTCAGCAAGGNAAAAGCCTTGGGTRTTCAATCCAGGTTGGGGAGGG |
| | | GATCCAAT |
| IL10 6Y135 | 20 | ACAAGCTGGACAACATACTGCTGACYGGGTCCCTGCTGGAGGACTTTAAGGTGAGAGCCCGGCT |
| PTPN3 | 21 | TAAAGGGCTTTTA[A/G]TCAGACCAGTTTCAATTC |
| PTPN15 | 22 | GATGAGAGAGGA[A/G]AATCAGGTTGGGCTGTT |
| INS8 | 23 | CCCACGTGTAGCCTC[A/G]TCCCCACCCAAGTG |
| INS1 | 24 | AGCCAGGAGGG[C/T]CCAGCAGCCCCCAGCCC |
| IGF2 10 | 25 | GGTCAAAGCCC[G/A]GGGCGAGCTGAGGCCC |
| TNF 9585 | 26 | AAAGTAGTGGGA[C/T]CTTTTCCAGGAAG |
| TNF 9367 | 27 | GAAAACTAAAGTCTGAGCTGCATAAGCTGTTTCTCCTA[C/T]AGGGGTGACTTGCTCTGA |
| | | TGCTAAACCT |
| TNF 10513 | 28 | GCTTAGAAAGAGAATTAAGGGCTCAGGGCTGG[G/A]CCTCAAGCTTAGAACTTTAAACGA |
| | | CACTTAGAAA |
| CTLA4 11R124 | 29 | TTTTGCCTGCTAACATTTCAGCTGGRTTTGAAGGCTTATATAAGGTTGGGGGG |
| CTLA4 11R204 | 30 | AGAAGCTCCCTGAGGAGCTGTCGTATTARTTAACTGCTGGAGGAGAAGAAGGAGGATTGGATAAG |
| | | ATAATGG |
| CTLA4 11R386 | 31 | GCATTAGGCCCGTATTCCACARAGTGTCCTCTACTGTGCTGAGCTATATGGA |
| CTLA4 11Y437 | 32 | TATGGACAGTGGGAAATCATAAAGTGYGGGAATAGGCAATCACCATATTCC |
| CTLA4 11Y540 | 33 | GCATTAACTGCATTTTGTCCAGTCATCTTTYAATCTAAGTGCATATCCCATATCACTGGCATATCACAGGTTC |
| CTLA4 12Y232 | 34 | GCTTGAAAAGTTCCCTTTAGAAAGAAAAACATGTYTCTCCTCATATGGAAGGTTTGAATCTCTTGGATCATTT |
| | | TGGCTGAC |
| CTLA4 12K291 | 35 | GGATCATTTTGGCTGACTTTTTTTGGACCKTTTCCAACTCTATTTTGTCTTTGTTAAGGCTTTTAAGA |
| IFNg 5M532 | 36 | AAATTATCAATGTGCTCTATGGMTGAGGACTCAACAATTTACAAAGGCAAAGGAT |

TABLE 6

Sequence Map for Haplotypes.
The SNPs below form the haplotypes shown in Table 3. Taking the SNPs from left to
right in Table 3 corresponds to the SNPs in bold going top to bottom in this Table.

| | | | |
|---|---|---|---|
| | | SNP ref/ | |
| | Generic | SEQ ID | |
| SNP | code | NO: | Sequence |

| | | | |
|---|---|---|---|
| CTLA411R124 | | 29 | TTTTGCCTGCTAACATTTCAGCTGGRTTTGAAGGCTTATATAAGGTTGGGGGG |
| CTLA411R204 | | 30 | AGAAGCTCCCTGAGGAGCTGTCGTATTARTTAACTGCTGGAGGAGAAGAAGGAGGATTGGATAAGATAATGG |
| CTLA411R269 | | 36 | GATAAGATAATGGGAGAAAATAGGCATTGGAACARCATGAGTAAAGTTGATGAGA |
| CTLA411M291 | | 37 | ATGAGTAAAGTTGATGAGATM(291)TGTAAGAGGTATGTTGR(308)ACAAAAAGAGGAAGGGGGCA |
| CTLA411R308 | | 38 | ATGAGTAAAGTTGATGAGATM(291)TGTAAGAGGTATGTTGR(308)ACAAAAAGAGGAAGGGGGCA |
| CTLA411R364 | | 39 | AAGAAATGCTGGAAGCCAGGCTAAAAAGAGARGCATTAGGCCCGTATTCCA |
| CTLA411R386 | | 31 | GCATTAGGCCCGTATTCCACARAGTGTCCTCTACTGTGCTGAGCTATATGGA |
| CTLA411Y437 | | 32 | TATGGACAGTGGGAAATCATAAAGTGYGGGAATAGGCAATCACCATATTCC |
| CTLA411Y540 | | 33 | GCATTAACTGCATTTTGTCCAGTCATCTTTYAATCTAAGTGCATATCCCATATCACTGGCATATCACAGGTTC |
| CTLA412M78 | | 40 | AGTACATGAAAACTCCTCMGTATTAAGCGAGGTGGTCCCCAATG |
| CTLA412Y232 | | 34 | GCTTGAAAAGTTCCCTTTAGAAAGAAAAACATGTYTCTCCTCATATGGAAGGTTTGAATCTCTTGGATCATTT |
| | | | TGGCTGAC |
| CTLA412K291 | | 35 | GGATCATTTTGGCTGACTTTTTTTGGACCKTTTCCAACTCTATTTTGTCTTTGTTAAGGCTTTTAAGA |
| CTLA412K375 | | 41 | AGCCAGAGGCAAATTCATTKATTTCCCGTGATTTGGGTATTTTCTCTCAACAAAATGCTAA |
| CTLA413R176 | | 42 | TATGGACTAAAGCTGTCATGGGTCAAGGRCTCAGACCAGCAGCTTAGCAGCTTTGGAGATGTG |
| CTLA413Y435 | | 43 | GAGGTTATCTTTTCGACGTAACAGCTAAACCCAYGGCTTCCTTTCTCGTAAAACCAAAACAAAAAGGCTTT |
| IFNg 4R430 | | 44 | TAAAGATAGGGAAACTGAATCATRGGAGAGTTAGGATGCTTCCTCAGAATCACAT |
| IFNg 5M509 | | 45 | TTCCTTTTTTACTTACTTCTGACCACAAAMAAATTATCAATGTGCTCTA |
| IFNg 5M532 | | 36 | AAATTATCAATGTGCTCTATGGMTGAGGACTCAACAATTTACAAAGGCAAAGGAT |
| IFNg 15Y221 | | 46 | CGCCACTTGAATGTGTCAGGTGATATGACYTGTGTCCTGATTAACACATAGCATTTCTTCT |
| IFNg 15W376 | | 47 | ATAATTTCATAATGATTCATGCWGTGTCAAACTTTTTCTGGGGTAAATGAACTA |
| IL-10 13Y85 | | 14 | ACAGACGCCATAGTCTTCCTATAAACTCAGTYCTTTAAGACATTATCCTTAAACTCTAAAAGATCATGCTG |
| IL-10 14R553 | | 15 | GTCACAGTTTACTGAGCACTTATTTTGAGCCAGCCRGTGCTAGTTCTGTACATGTCAGCCATAGGGTAT |
| IL-10 1R105 | | 16 | GCTCTTCCTAGTTACTGTCTTCACTGGGGAGGTAR(105)GAAAAGCTCCTR(117)TAGAAGGAGAAGGTCA |
| | | | AGGTACATCAAGGGACCC |
| IL-10 1R117 | | 17 | GCTCTTCCTAGTTACTGTCTTCACTGGGGAGGTAR(105)GAAAAGCTCCTR(117)TAGAAGGAGAAGGTCA |
| | | | AGGTACATCAAGGGACCC |
| IL-10 1R218 | | 18 | CCGCCCTCTCCTTTCCTTATTAGAGGTARAGCAACTTTCCTCACTGCACCTGCCTACCGCCCCTGC |
| IL-10 1K362 | | 48 | AAGGAGGGAAGGGACAGGTAAGAGAAAAAAAAAGCGGGGGGGKGGGGGGCCTGCAGTCCAGTCTTCATGGAAT |
| | | | CCTGACTTAACT |
| IL-10 2R420 | | 19 | ACTTGGGGAAACTGAGGCTCTTCCCAGTTCAGCAAGGNAAAAGCCTTGGGTRTTCAATCCAGGTTGGGGAGGG |
| | | | GATCCAAT |
| IL-10 3M171 | | 49 | AAAAGCTGGAAAGTTATTTTAAAACMGAGAGAGAGGTAGCTCATCCTAAAATAGCTGTAATG |
| IL-10 4Y100 | | 50 | AGCCAGCCGACACCAGAGCACCCTACYTGAGGACGACTGCACCCACTTCCCAGCCAGCCTGCCC |
| IL-10 6Y135 | | 20 | ACAAGCTGGACAACATACTGCTGACYGGGTCCCTGCTGGAGGACTTTAAGGTGAGAGCCCGGCT |
| IL-10 6R426 | | 51 | CCCCAACGCTYTTGCCTTTRGTTACCTGGGTTGCCAAGCCCTGTCGGAG |
| IL-10 9R210 | | 52 | AGCTGTCCCCCAAGTGCCAGGGACACRGGAGCTGGGAGCCGTGGCATTAACACTTT |
| IL-10 10S308 | | 53 | CCGCACCCTCTTCCCAGAACAGGCGGCCTCSGCCCTCTGCGGGGCTGAGCCC |
| IL-10 11R124 | | 13 | CGCTTTTTAGGCCAACCCCGCRTCGCCTCTCCCAAGGCGACTGG |
| IL-12b 1Y90 | | 12 | TTTCCCTACAGCCAGGCACGACTTTTTACCCTACYATTGTACACAAAACAGACATATC |
| IL-12b 1M115 | | 54 | ATTGTACACAAAACAGACATATCMGATATTTCCTTTATCTCTTC |
| IL-12b 2Y146 | | 55 | CTTATTCTTCTTATGATTTAGTCAGYGGYTTCTAACCAYGTGTCAGAGAACATGGATGCTCTCTGAGAT |
| IL-12b 2Y190 | | 56 | CATGGATGCTCTCTGAGATGGATGGAGATGTTYCAGGATGAGATGAAATGATAA |
| IL-12b 2W232 | | 57 | TAAATATCTCTACCTAATTCAGAWGTAGGGTACAGTTTTCACATTCTAAATATTTG |
| IL-12b 2M407 | | 10 | GGGTATATCAATATTTTAGGGTCTTCTCCCAAAGAACCTCTTGATTTTCAGMGCTTATGGGCTTGAACATGGG |
| | | | TTAAACCAGTGGTTCTCAAAGTGTGGTCT |
| IL-12b 3Y82 | | 58 | TTTAACAAGGCTTCCAGGTTACTTTGATGTGYACTCAAGCTTGAGAATCACTGG |
| IL-12b 3R196 | | 11 | AAACAAGGAGAGAAACTAAACCTGGCCACCAGATCATTGCCRTAATTTGAAATCACCTCTAATTGTCTCCCAC |
| | | | CACCACCA |
| IL-12b 3R462 | | 59 | TCTCGCTCAGAGCCTTTTACATAGTCARTACCAAGTATATAATTGCTAAATGTTGATCCCA |
| IL-12b 10R105 | | 60 | TCTCCACTCCCTGTGCTCTCCAGTTTATRTTGTAGAGTTGGACTGGCACCCTGATGCCCCCG |
| IL-12b 12Y142 | | 61 | TTTCTGAAATGTGAGGCAAAGAATYATTCTGGACGTTTCACATGCTGGTGGCTGACGG |
| IL-4 25Y336 | | 3 | CCTTTGGGTATATTTCCAGAAGTAGAATTACTGGATCATGTAGCATTTGTATTTTYAGTTTTTTGAGGATTTT |
| | | | TCATACTGTTTTCCATAGTGGCTGCACCAG |
| IL-4 22Y15 | | 62 | AGGTCATCTTGTGAAGGACAGAATCCAYGTGAGTGTATGAGGAAGGCCCTGCAACCATATT |
| IL-4 13S97 | | 1 | GCTAGGCGTGAGATCAGAGGAAGCTTCTGGAAGAGGSTGCAGTTGAGCTGGGCCATGGACACAA |
| IL-4 12M397 | | 63 | GGGCAGCACTCTCCAGTTAGCTCCCCCACCCMCCTCCATGGGAGGTGGCAAGTGTCTGCAAAG |
| IL-4 8R458 | | 2 | TCAAACTTAGTATTGATAAATTGAACTCCTGATCTTCTGCTCAACCTCCARCACTGCTCTGCGCTCAATTTTC |
| | | | TGGGCACCAGCCCTCTCCCAAAAGGCT |
| IL-4 7S246 | | 64 | CCTTAAGAATCAGGTGACAGGCTCAGCAAGGGGATSAATGTCCCCAATTCTTCCATTTGGCAC |
| IL-4 2M351 | | 5 | AACCTTGGATATTGTGTGTTAATTTCTGTATTGAAAAGTGAGGGTTCACTTCATTTGTACTACCCCTTCCAMA |
| | | | TTTTTTATAGTGAATTTATTTTCAGATCTTGTATTACC |
| IL-4 1K110 | | 4 | TGATTGCCACTTCTGGATGTTTCATATAAATGGAATCATGTAGCCTTTTGTATCTGKCTTCTTTCACTTACCC |
| | | | TAGTGTTTCTAAGGTTCATCCATATTGTAGCG |
| IL-6 6K372 | | 7 | AGCTAAACCACTAAGCCACCAGGGCTGCCCCCAAGTCATATTTTCTAAAACATAKATATATATGAGAAAAAGC |
| | | | AATCCCACACTACAGAGGCTTTTTG |
| IL-6 6R431 | | 6 | ATATGAGAAAAAGCAATCCCACACTACAGAGGCTTTTTGCAAGCATCACAGTGGRGCTGGGAGAGGTGGCTTC |
| | | | ATTCAGCGCAGGAGAGAGGACTCGGCTGGCAGTGTC |
| IL-6 7S166 | | 65 | AAGAAAACCTAGGGCAAGCGTGATTCAGAGCCTCAGAGSCTTGTCTGTGTTTGGAGATTCCTTCTCAGGCACC |
| | | | TCTG |
| IL-6 7R485 | | 66 | ACATGACACAGAGATCCAAGTCTTCACCAGGGCCCCTGCRCAGAGAGCAGGGCTGACGCTG |
| IL-6 8R289 | | 67 | ACGTCTTAGGTTTTCACAAATATGAATTAACTGRAATGCTAAATCCTAGCCCGCTAATCTGGTA |
| IL-6 8W328 | | 68 | TAGCCCGCTAATCTGGTAATTAAAGTWTTTTTTTAATCATAGCCTTAGCTTCTC |
| IL-6 10Y257 | | 69 | CCCGGGACCCCTGGCAGGAGATTCCAAGGATGAYGCCACTTCAAATAGTCTACCACTCACCT |
| IL-6 18R120 | | 70 | GCAGTCGCAGGATGAGTGGCTGAAGCACACAACAATTCACCTCATCCTGCRGAGTCTGGAGGATTTCCTGCAG |
| | | | TTCAGTCTGA |
| IL-6 20R191 | | 8 | CCAGCCCCTGTACACACTTTTATGGACRTAGGAGAAGGGACTTCCCAAA |
| IL-6 20R240 | | 9 | CCAGCTAGAAGGTAAGGCACAGRCCCAGATTTTAAATCCAGGTCTAATTG |
| IL-6 20R412 | | 71 | GTAAAGATGCAATCAAAAGCCTTTGAAATGACAACCACTTATRTAAGACCTAGCAATGTGCACTTCCAAACA |
| | | | TTA |
| IGF 10 | R | 25 | GGTCAAAGCCC[G/A]GGGCGAGCTGAGGCCC |
| IGF 4 | R | 72 | GCTCCTATGCC[A/G]GTAACCACCCCC |
| IGF 3 | M | 73 | CCCCCAAACA[A/C]CCTAAAATCCATC |
| IGF 2 | R | 74 | CCTCTTGACcAGGGGC[C/T]ATTCCATCGGGTCC |
| IGF 1 | R | 75 | GGGGACGCCCTC[G/A]TGGTCAGGCCTGGCC |
| INS 8 | R | 23 | CCCACGTGTAGCCTC[A/G]TCCCCACCCAAGTG |
| INS 5 | Y | 76 | CTGAGGTCCCTTCC[C/T]GGGCCACCCCCTCCCC |
| INS 9 | R | 77 | GTGGTCAGGCCAC[A/G]CCGGCGCCGAGCCCCA |
| INS 4 | R | 78 | GGCANGGGGTGG[A/G]GTGGGCGGGGCGCGC |
| INS 10 | R | 79 | AGCTCCCTTCACGC[A/G]GGGAGTCTCAGAATGT |
| INS 1 | Y | 24 | AGCCAGGAGGG[C/T]CCAGCAGCCCCCAGCCC |
| IL1a 8619 | K | 80 | AGGAAACCTTCAACATTTATCTGCCAAGAGTCTGACGTG/T]GTACCACCTGAACTGGGCCAG |
| IL1a 10084 | M | 81 | CTAGGAGAGGAGGCAGATACATATGCAGATAACACAAGGGAGTGA/C]AAAGAAGAATGGGGAAAATG |
| | | | CTGAGTGTGGGCTAAGTCATTCATTAAGCTTCTCAAGAAGCACAAAGCAGTGGTGA |
| IL1a 11235 | S | 82 | TGTGTTACCAAAGCTAATGTGGTCATTAAAACAA[C/G]TGCAGAGATGTAACAAACAGAATTACATTC |
| | | | TCATTATCTTGTTTG |
| IL1a 12227 | Y | 83 | AAAGCAGTTACATACTACTCATAAGCTATGTT[T/C]CTCCAGATAATAACTATGCTCCTTTGTAAGTT |
| | | | ACT |
| IL1a E7x221 | Y | 84 | GCCTTGACTCTGGAGTCTATAACTTGTGAYGTGTTGACAGTCCACGTGTACTATGTACA |
| IL1a E7x225 | R | 85 | TTGACAGTCCACGTGTACTATGTACATGGARGAGTCCAATCCTTTACTCATAGTCACTTGCTGA |
| PTPN 11 | R | 86 | AAATGTACAAAAAG[C/T]AAAATAAGACAAACAC |
| PTPN 12 | R | 87 | GGATACATTTAGC[C/T]AATCAGTTATGACTA |
| PTPN 13 | R | 88 | CAAAAGAAAC[A/G]GAGTAATAGGGG |
| PTPN 15 | Y | 22 | GATGAGAGAGGA[A/G]AATCAGGTTGGGCTGTT |
| PTPN 1 | W | 89 | ATGAGAATGTATAA[A/T]GGGAGGTTTGCTCTAT |
| PTPN 2 | R | 90 | AATCTGAAGAACTA[C/T]GAAGTGTTAACTAGGTA |
| PTPN 3 | R | 21 | TAAAGGGCTTTTA[A/G]TCAGACCAGTTTCAATTC |
| PTPN 7 | R | 91 | TTTTTTTCAGCT[G/A]TTTAAAACTGTGAAATA |
| PTPN 5 | S | 92 | CCCCAGCCCT[C/G]GGGAGAGATA |
| PTPN 4 | R | 93 | ATAGTGTTT[A/G]GAATCATAATT |
| PTPN 9 | R | 94 | GTTTTGGGGTA[C/T]CCAGCTTGCTCAGGCA |
| TNF 3 | W | 95 | GCCTCTTTTGGCT[A/T]CATAACTCTCCTGCA |
| TNF 4 | S | 96 | CCGAGGGGGGC[G/A]AGTAGGAAGTAT |
| TNF 6547 | M | 97 | TTGGAGCCTTCGCTCTGTAGAAAAATCC[A/C]GAAAAAAAAAATTGGTTTCAAGACCTTTTC |
| TNF 7178 | W | 98 | AAACCTCTTTTCTC[T/A]GAAATGCTGTCT |
| TNF 8647 | M | 99 | CCAGGGCTCTAC[C/A]GTCTCCCCACTGG |
| TNF EXON1AB | R | 100 | GGG CTC CAG AAG GTG CTT CTG CCT CAG CCT CTT CTC CTT CCT CCT CRT CGC AGG |
| | | | GGC CAC CAC ACT CTT CTG |
| TNF 9367 | Y | 27 | GAAAACTAAAGTCTGAGCTGCATAAGCTGTTTCTCCTA[C/T]AGGGGTGACTTGCTCTGATGCTAAA |
| | | | CCT |
| TNF 9585 | Y | 26 | AAAGTAGTGGGA[C/T]CTTTTCCAGGAAG |
| TNF 1 | R | 101 | CAGACCTTAGAG[A/G]TGGTATGAGAGGGA |
| TNF 10252 | W | 102 | GGAGACCCCAG[A/T]GGGGACCGAGG |
| TNF EXON4AB | W | 103 | AAC CTA CTC TCT GCC ATC AAG AGC CCT TGC CAA AGG GAG ACC CCA GAG GGG ACC |
| | | | GAG GCC AAG CCC TGG TAC GAG CCC ATC TAC CTG GGA GGG GTC TTC CAA CTG GAG |
| | | | AAG |
| TNF 10411 | R | 104 | TACTTTGGAATCATTGCCCTGTAAGGGG[G/A]TAGGACGTCCATTCTTGCCCAAACCGACCCTTTGAT |
| | | | CACTCACTTCCTCTGACCCCTCACCCCCTTCAG |
| TNF 10513 | R | 28 | GCTTAGAAAGAGAATTAAGGGCTCAGGGCTGG[G/A]CCTCAAGCTTAGAACTTTAAACGACACTTAG |
| | | | AAA |
| RANTES 15W74 | | 105 | CCTGAGAGAGGATTTTTTTAWTTTTAATTTTTTTAAGATTTATTTGA |
| RANTES 15S358 | | 106 | TTCCCAGATGACTGAGTGGCTGAGCTTSACTGAAAGACGGAGAAACAGAGGCTCA |
| RANTES 17Y105 | | 107 | CAGTCTATCCAAGATAATGTACCCAGCACAAYACCCCATGTATAATGGCAATGAGT |
| RANTES 17R307 | | 108 | GCCCTGTGGACCCTCTGGGGGGGGCAGRGGGGGATGAGGAAGGGACACCTTTTGTTCCAGAG |

TABLE 7
SNP codes

| IUB/GCG | Meaning | Complement |
|---|---|---|
| A | A | T |
| C | C | G |
| G | G | C |
| T/U | T | A |
| M | A or C | K |
| R | A or G | Y |
| W | A or T | W |
| S | C or G | S |
| Y | C or T | R |
| K | G or T | M |

TABLE 8
Taqman Assay Identification, Primer and Reporter Sequences

| | Forward | | | Reporter 1 | Reporter 1 |
|---|---|---|---|---|---|
| Assay ID | Primer Name | Forward Primer Seq (5′-3′) | Assay ID | Name (VIC) | Sequence (5′-3′) |
| CTLA4 11R124 | CTLA4 11R124F | GGTTGCTTTTGCCTGCTAACA | CTLA4 11R124 | CTLA4 11R124V | TTTCAGCTGGATTTGAA |
| CTLA4 11R204 | CTLA4 11R204F | AGGGCCTCAGGAGAAGCT | CTLA4 11R204 | CTLA4 11R204V | CTGTCGTATTAATTAACTG |
| CTLA4 11R269 | CTLA4 11R269F | GAGGAGAAGAAGGAGGATTGGAT | CTLA4 11R269 | CTLA4 11R269V | CATTGGAACAACATGAG |
| | | AAG | | | |
| CTLA4 11M291 | CTLA4 11M291F | ATGGGAGAAAATAGGCATTGGAA | CTLA4 11M291 | CTLA4 11M291V | CATACCTCTTACATATCTCA |
| | | CA | | | |
| CTLA4 11R308 | CTLA4 11R308F | ATGGGAGAAAATAGGCATTGGAA | CTLA4 11R308 | CTLA4 11R308V | TCCTCTTTTTGTTCAACATA |
| | | CA | | | |
| CTLA4 11R364 | CTLA4 11R364F | GGCATGTGAAGAAATGCTGGAA | CTLA4 11R364 | CTLA4 11R364V | CTAAAAAGAGAAGCATTAGG |
| CTLA4 11R386 | CTLA4 11R386F | TGCTGGAAGCCAGGCTAAAA | CTLA4 11R386 | CTLA4 11R386V | TTCCACAAAGTGTCCTC |
| CTLA4 11Y437 | CTLA4 11Y437F | CTGAGCTATATGGACAGTGGGAA | CTLA4 11Y437 | CTLA4 11Y437V | CTATTCCCGCACTTTA |
| | | AT | | | |
| CTLA4 11Y540 | CTLA4 11Y540F | TCTCCTAGAAGTCCCTTAAGGCA | CTLA4 11Y540 | CTLA4 11Y540V | CACTTAGATTGAAAGATG |
| | | TT | | | |
| CTLA4 12K291 | CTLA4 12K291F | CTCATATGGAAGGTTTGAATCTC | CTLA4 12K291 | CTLA4 12K291V | CTGACTTTTTTTGGACCGAA |
| | | TTGGA | | | |
| CTLA4 12K375 | CTLA4 12K375F | TGAATTCTTTCCTAATCTGCAAG | CTLA4 12K375 | CTLA4 12K375V | AATTCATTGATTTCCC |
| | | CCA | | | |
| CTLA4 12M78 | CTLA4 12M78F | GCATATCACAGGTTCTCAAGAAA | CTLA4 12M78 | CTLA4 12M78V | ATGAAAACTCCTCAGTATTA |
| | | TGTC | | | |
| CTLA4 12Y232 | CTLA4 12Y232F | CTTGGATTTTATGCTTGAAAAGT | CTLA4 12Y232 | CTLA4 12Y232V | ATATGAGGAGAGACATGTT |
| | | TCCCTTT | | | |
| CTLA4 13R176 | CTLA4 13R176F | GCAGGGCTTTTATTAATGATGTC | CTLA4 13R176 | CTLA4 13R176V | TCAAGGACTCAGACCAG |
| | | TATGG | | | |
| CTLA4 13Y435 | CTLA4 13Y435F | AGTGTTTGAGGTTATCTTTTCGA | CTLA4 13Y435 | CTLA4 13Y435V | AAAGGAAGCCGTGGGTT |
| | | CGTA | | | |
| IFNg 4R430 | IFNg 4R430F | GTATCAGTCCCATTTTAAAGATA | IFNg 4R430 | IFNg 4R430V | CCTAACTCTCCTATGATTC |
| | | GGGAAACT | | | |
| IFNg 5M509 | IFNg 5M509F | AGGTTTGAGTTCCCTTAGAATTT | IFNg 5M509 | IFNg 5M509V | ACCACAAAAAAATTATC |
| | | CCTTTT | | | |
| IFNg 5M532 | IFNg 5M532F | GGTTTGAGTTCCCTTAGAATTTC | IFNg 5M532 | IFNg 5M532V | TGTTGAGTCCTCATCCATA |
| | | CTTTTTT | | | |
| IFNg 15Y221 | IFNg 15Y221F | AGACGCCACTTGAATGTGTCA | IFNg 15Y221 | IFNg 15Y221V | CAGGACACAGGTCATAT |
| IFNg 15W376 | IFNg 15W376F | GACTGTACCCAATGGAAAACAAT | IFNg 15W376 | IFNg 15W376V | TTTGACACAGCATGAAT |
| | | TAATTTGT | | | |
| IL-10 4Y100 | IL-10 4Y100F | CAGCCGACACCAGAGCA | IL-10 4Y100 | IL-10 4Y100V | TCGTCCTCAGGTAGGG |
| IL-10 6R426 | IL-10 6R426F | GCTCTTCCGCCCAGTCA | IL-10 6R426 | IL-10 6R426V | CCCAGGTAACTCTAAAG |
| IL-12b 10R105 | IL-12b 10R105F | TCATGAAGCTCACAATCCAGTTC | IL-12b 10R105 | IL-12b 10R105V | CAACTCTACAATATAAAC |
| | | TC | | | |
| IL-12B 12Y142 | IL-12B 12Y142F | GAATTTTTGTTCTTTTCAAATCC | IL-12B 12Y142 | IL-12B 12Y142V | CCAGAATGATTCTTTG |
| | | AGAATCCAAA | | | |
| IL-6 18R120 | IL-6 18R120F | TGGCTGAAGCACACAACAATTC | IL-6 18R120 | IL-6 18R120V | CATCCTGCAGAGTCT |
| RANTES 13W74 | RANTES 13W74F | AGTCATATTCTCCCTGTTTCATA | RANTES 13W74 | RANTES 13W74V | AGAGGATTTTTTTAATTTT |
| | | GATGGA | | | |
| RANTES 13S358 | RANTES 13S358F | TGCTCTGCATGTACCATGTCATT | RANTES 13S358 | RANTES 13S358V | CTTTCAGTGAAGCTCA |
| | | TAAT | | | |
| RANTES 17Y105 | RANTES 17Y105F | CAGTTTCAGCCAAAGAAGGATAA | RANTES 17Y105 | RANTES 17Y105V | CAGCACAACACCCCA |
| | | CAG | | | |
| RANTES 17R307 | RANTES 17R307F | CCCTGTGGACCCTCTGG | RANTES 17R307 | RANTES 17R307V | CTCATCCCCCTCTGCC |
| RANTES 17M347 | RANTES 17M347F | TGAGGAAGGGACACCTTTTGTTC | RANTES 17M347 | RANTES 17M347V | CAGAGCCAGTACCCCA |
| | Reverse | | | Reporter 2 | Reporter 2 |
| Assay ID | Primer Name | Reverse Primer Seq (5′-3′) | Assay ID | Name (FAM) | Sequence (5′-3′) |
| CTLA4 11R124 | CTLA4 11R124R | CCCCTCCCCCCAACCTTATAT | CTLA4 11R124 | CTLA4 11R124M | TCAGCTGGGTTTGAA |
| CTLA4 11R204 | CTLA4 11R204R | TCTCCCATTATCTTATCCAATCC | CTLA4 11R204 | CTLA4 11R204M | CTGTCGTATTAGTTAACTG |
| | | TCCTT | | | |
| CTLA4 11R269 | CTLA4 11R269R | GGCTTCCAGCATTTCTTCACATG | CTLA4 11R269 | CTLA4 11R269M | CATTGGAACAGCATGAG |
| CTLA4 11M291 | CTLA4 11M291R | GGCTTCCAGCATTTCTTCACATG | CTLA4 11M291 | CTLA4 11M291M | CCTCTTACAGATCTCA |
| CTLA4 11R308 | CTLA4 11R308R | GGCTTCCAGCATTTCTTCACATG | CTLA4 11R308 | CTLA4 11R308M | CTCTTTTTGTCCAACATA |
| CTLA4 11R364 | CTLA4 11R364R | GTCCATATAGCTCAGCACAGTAG | CTLA4 11R364 | CTLA4 11R364M | AAAAGAGAGGCATTAGG |
| | | AG | | | |
| CTLA4 11R386 | CTLA4 11R386R | ACAGGCAAACAGACAGTTACAACA | CTLA4 11R386 | CTLA4 11R386M | CCACAGAGTGTCCTC |
| CTLA4 11Y437 | CTLA4 11Y437R | ACAGGCAAACAGACAGTTACAACA | CTLA4 11Y437 | CTLA4 11Y437M | CCTATTCCCACACTTTA |
| CTLA4 11Y540 | CTLA4 11Y540R | GAGAACCTGTGATATGCCAGTGAT | CTLA4 11Y540 | CTLA4 11Y540M | CACTTAGATTAAAAGATG |
| CTLA4 12K291 | CTLA4 12K291R | TCAGGTATTCTTAAAAGCCTTAA | CTLA4 12K291 | CTLA4 12K291M | CTGACTTTTTTTGGACCTAA |
| | | CAAAGACA | | | |
| CTLA4 12K375 | CTLA4 12K375R | AGCTCCATTTAGCATTTTGTTGA | CTLA4 12K375 | CTLA4 12K375M | CAAATTCATTTATTTCCC |
| | | GAGA | | | |
| CTLA4 12M78 | CTLA4 12M78R | AGGACCAGTGTTCATACTGTAAG | CTLA4 12M78 | CTLA4 12M78M | ATGAAAACTCCTCCGTATTA |
| | | AGA | | | |
| CTLA4 12Y232 | CTLA4 12Y232R | AAGTCAGCCAAAATGATCCAAGA | CTLA4 12Y232 | CTLA4 12Y232M | ATATGAGGAGAAACATGTT |
| | | GA | | | |
| CTLA4 13R176 | CTLA4 13R176R | CACATCTCCAAAGCTGCTAAGC | CTLA4 13R176 | CTLA4 13R176M | AAGGGCTCAGACCAG |
| CTLA4 13Y435 | CTLA4 13Y435R | GCACCTGAATAGAAAGCCTTTTT | CTLA4 13Y435 | CTLA4 13Y435M | AAAGGAAGCCATGGGTT |
| | | GT | | | |
| IFNg 4R430 | IFNg 4R430R | GGCTATGTGATTCTGAGGAAGCAT | IFNg 4R430 | IFNg 4R430M | TAACTCTCCCATGATTC |
| IFNg 5M509 | IFNg 5M509R | ACCTCCATCCTTTGCCTTTGTAA | IFNg 5M509 | IFNg 5M509M | CACAAACAAATTATC |
| | | AT | | | |
| IFNg 5M532 | IFNg 5M532R | ACCTCCATCCTTTGCCTTTGTAA | IFNg 5M532 | IFNg 5M532M | TTGAGTCCTCAGCCATA |
| | | AT | | | |
| IFNg 15Y221 | IFNg 15Y221R | GGGTACAGTCATAGTTGTCAGTG | IFNg 15Y221 | IFNg 15Y221M | CAGGACACAAGTCATAT |
| | | GTA | | | |
| IFNg 15W376 | IFNg 15W376R | AACTCATTAGAGTATATAGTTCA | IFNg 15W376 | IFNg 15W376M | TTGACACTGCATGAAT |
| | | TTTACCCCAGAA | | | |
| IL-10 4Y100 | IL-10 4Y100R | AGGCTGGCTGGGAAGTG | IL-10 4Y100 | IL-10 4Y100M | TCGTCCTCAAGTAGGG |
| IL-10 6R426 | IL-10 6R426R | CCTCCTCCAAGTAAAACTGGATC | IL-10 6R426 | IL-10 6R426M | CCCAGGTAACCCTAAAG |
| | | AT | | | |
| IL-12b 10R105 | IL-12b 10R105R | CAGGTGAGGACCACCATTTCTC | IL-12b 10R105 | IL-12b 10R105M | CAACTCTACAACATAAAC |
| IL-12B 12Y142 | IL-12B 12Y142R | GCCACCAGCATGTGAAACG | IL-12B 12Y142 | IL-12B 12Y142M | TCCAGAATAATTCTTTG |
| IL-6 18R120 | IL-6 18R120R | CAGACTGAACTGCAGGAAATCCT | IL-6 18R120 | IL-6 18R120M | CATCCTGCGGAGTCT |
| RANTES 13W74 | RANTES 13W74R | CCCTCCCCTCTATTCTCTCTCAA | RANTES 13W74 | RANTES 13W74M | AGAGGATTTTTTTATTTTT |
| | | AT | | | |
| RANTES 13S358 | RANTES 13S358R | CTCCTCTGAGCCTCTGTTTCTC | RANTES 13S358 | RANTES 13S358M | CTTTCAGTCAAGCTCA |
| RANTES 17Y105 | RANTES 17Y105R | GTAGACTCCTGTACTCATTGCCA | RANTES 17Y105 | RANTES 17Y105M | CAGCACAATACCCCA |
| | | TT | | | |
| RANTES 17R307 | RANTES 17R307R | ACTGGCTCTGGAACAAAAGGT | RANTES 17R307 | RANTES 17R307M | TCATCCCCCCCTGCC |
| RANTES 17M347 | RANTES 17M347R | GGAGTGGATAGGGTAGGCTCTTA | RANTES 17M347 | RANTES 17M347M | AGAGCCAGTCCCCCA |

TABLE 9
Sequenom Primers, Pools and amplicon length

| | | | | |
|---|---|---|---|---|
| WELL | SNP_ID | 2nd-PCRP | 1st-PCRP | AMP_LEN |

| | | | | |
|---|---|---|---|---|
| W1 | IL-4_7S246 | ACGTTGGATGAAGAATCAGGTGACAGGCTC | ACGTTGGATGGGAAGAGCTCAGAGTAGATG | 106 |
| W1 | IL-12B_10R105 | ACGTTGGATGTGAGGACCACCATTTCTCCG | ACGTTGGATGACAATCCAGTTCTCCACTCC | 110 |
| W1 | IL-12B_02M407 | ACGTTGGATGCCACACTTTGAGAACCACTG | ACGTTGGATGGTCTTCTCCCAAAGAACCTC | 99 |
| W1 | IL-12B_03Y82 | ACGTTGGATGTAACAAGGCTTCCAGGTTAC | ACGTTGGATGGCTCCAAACTCAAAGGTTAC | 111 |
| W1 | IL-12B_02Y190 | ACGTTGGATGATGCTCTCTGAGATGGATGG | ACGTTGGATGATGTGAAAACTGTACCCTAC | 110 |
| W1 | IL-12B_01Y90 | ACGTTGGATGCAGCCAGGCACGACTTTTTA | ACGTTGGATGATGTCAGCTTGTACCAAGGG | 111 |
| W1 | TNFexon4aAB | ACGTTGGATGACTCGGCAAAGTCCAGATAG | ACGTTGGATGGGTCTTCCAACTGGAGAAGG | 94 |
| W1 | IL-4_8R458 | ACGTTGGATGCTGGTGCCCAGAAAATTGAG | ACGTTGGATGGAACTCCTGATCTTCTGCTC | 81 |
| W1 | IL-10_1R117 | ACGTTGGATGGTCCCTTGATGTACCTTGAC | ACGTTGGATGTGCTCTTCCTAGTTACTGTC | 100 |
| W1 | IL-10_1R218 | ACGTTGGATGCGCCCTCTCCTTTCCTTATT | ACGTTGGATGTGTGTGTGTGTTTGAGGGTG | 106 |
| W1 | IL-4_25Y336 | ACGTTGGATGGAATTACTGGATCATGTAGC | ACGTTGGATGAAACTGGTGCAGCCACTATG | 102 |
| W1 | IL-10_4Y100 | ACGTTGGATGACTGCTCTGTTGCTGCCTG | ACGTTGGATGTGGGAAGTGGGTGCAGTCG | 111 |
| W1 | IL-12B_12Y142 | ACGTTGGATGGATCTTTCTGAAATGTGAGGC | ACGTTGGATGCAAATCAGTACTGATTGCCG | 99 |
| W1 | TNF10252 | ACGTTGGATGATCAAGAGCCCTTGCCAAAG | ACGTTGGATGTTCTCCAGTTGGAAGACCCC | 115 |
| W1 | TNF7178 | ACGTTGGATGATCTGCACCTTCAACGAAGC | ACGTTGGATGAAAATTCTCCCCTCCCAGAC | 102 |
| W1 | IL-12B_03R196 | ACGTTGGATGTGGTGGTGGGAGACAATTAG | ACGTTGGATGGGAGAGAAACTAAACCTGGC | 92 |
| W1 | TNF10411 | ACGTTGGATGAGTGAGTGATCAAAGGGTCG | ACGTTGGATGGGCAGGTGTACTTTGGAATC | 101 |
| W1 | IL-10_14R553 | ACGTTGGATGACAGCCGATGAGATGTTGAC | ACGTTGGATGAATCCCATACCCTATGGCTG | 119 |
| W1 | IL-10_11R124 | ACGTTGGATGTCGCTAGCCACGCTTTTTAG | ACGTTGGATGTGAAGGATGGACCCAGGCAA | 107 |
| W1 | IL-6_20R191 | ACGTTGGATGCTTCTAGCTGGGTGACTTTG | ACGTTGGATGTATGATGCTCAATCCCAGCC | 99 |
| W2 | IL-10_9R210 | ACGTTGGATGAAGTGTTAATGCCACGGCTC | ACGTTGGATGGAGTCTGGGCCCTTTTTCAG | 101 |
| W2 | IL-10_10S308 | ACGTTGGATGCACCCTCTTCCCAGAACAG | ACGTTGGATGGGGAGCAGGCCCTGCCCG | 106 |
| W2 | TNFexon1AB | ACGTTGGATGTTCTGCCTCAGCCTCTTCTC | ACGTTGGATGATCACTCCAAAGTGCAGCAG | 97 |
| W2 | IL-4_2M351 | ACGTTGGATGGTGAGGGTTCACTTCATTTG | ACGTTGGATGGCACAGGTAATACAAGATCTG | 99 |
| W2 | IL-12B_02Y146 | ACGTTGGATGTCTCCATCCATCTCAGAGAG | ACGTTGGATGCTTCTTATGATTTAGTCAG | 92 |
| W2 | IL-6_8R289 | ACGTTGGATGTTACCAGATTAGCGGGCTAG | ACGTTGGATGGAAGCTCAGGTCTAAACGTC | 100 |
| W2 | IL1a10084 | ACGTTGGATGGAATGACTTAGCCCACACTC | ACGTTGGATGGGAGGCAGATACATATGCAG | 99 |
| W2 | IL-6_7S166 | ACGTTGGATGTGTTTTGAGTCCAGAGGTGC | ACGTTGGATGAAGAAAACCTAGGGCAAGCG | 108 |
| W2 | IL-4_22Y152 | ACGTTGGATGCTCTCCCTACTGATTTCCTC | ACGTTGGATGAATATGGTTGCAGGGCCTTC | 101 |
| W2 | IL-6_20R240 | ACGTTGGATGTCACCCAGCTAGAAGGTAAG | ACGTTGGATGGGGACCCTAAAGGTTAAGAG | 109 |
| W2 | IL-6_7R485 | ACGTTGGATGACTCTCTTGCTCACCTCTTC | ACGTTGGATGAGATCCAAGTCTTCACCAGG | 109 |
| W2 | IL-6_18R120 | ACGTTGGATGCTGAACTGCAGGAAATCCTC | ACGTTGGATGTATCTTGCAGTCGCAGGATG | 104 |
| W2 | IL-6_20R412 | ACGTTGGATGTTGGAAGTGCACATTGCTAG | ACGTTGGATGAGGGAATGCATGTAAAGATG | 100 |
| W2 | IL-12B_01M115 | ACGTTGGATGATGTCAGCTTGTACCAAGGG | ACGTTGGATGGGCACGACTTTTTACCCTAC | 105 |
| W2 | TNF6547 | ACGTTGGATGCAGAATGGAGGCAAAATGGG | ACGTTGGATGTGTCTTCTTTGGAGCCTTCG | 107 |
| W2 | IL-4_1K110 | ACGTTGGATGGCCACTTCTGGATGTTTCAT | ACGTTGGATGCGCTACAATATGGATGAACC | 120 |
| W2 | IL1a11235 | ACGTTGGATGACCGTGTGTGTTACCAAAGC | ACGTTGGATGCTGTCAAACAAGATAATGAG | 110 |
| W2 | IL-10_13Y85 | ACGTTGGATGTACAGACGCCATAGTCTTCC | ACGTTGGATGCCTTAGTCTTGAAAACCAGC | 108 |
| W2 | IL-6_6R431 | ACGTTGGATGAGCAATCCCACACTACAGAG | ACGTTGGATGCTCTCCTGCGCTGAATGAAG | 98 |
| W2 | IL1aE7x221 | ACGTTGGATGTACATAGTACACGTGGACTG | ACGTTGGATGCTTTCGGTTACTGGAAACCC | 98 |
| W3 | IL-4_12M397 | ACGTTGGATGCTGGATATTGGTGCTTTGGG | ACGTTGGATGCTTTGCAGACACTTGCCACC | 100 |
| W3 | IL-10_6R426 | ACGTTGGATGACTGGATCATCTCCGACAGG | ACGTTGGATGCAGCTCTTCCGCCCAGTCA | 117 |
| W3 | TNF8647 | ACGTTGGATGCTAATATACAAGGCCCCAGG | ACGTTGGATGCTTTCAGTGCTCATGGTGTG | 101 |
| W3 | IL-4_13S97 | ACGTTGGATGAGATCAGAGGAAGCTTCTGG | ACGTTGGATGCTATACCTCCTAGGCCAAAG | 107 |
| W3 | IL-10_6Y135 | ACGTTGGATGGCAGCAAATGAAGGACAAGC | ACGTTGGATGGCTCTCACCTTAAAGTCCTC | 92 |
| W3 | IL-12B_02W232 | ACGTTGGATGTTACTATCCAGGGTTTGTGC | ACGTTGGATGCAGGATGAGATGAAATGAT | 113 |
| W3 | IL-10_1R105 | ACGTTGGATGTGCTCTTCCTAGTTACTGTC | ACGTTGGATGGTCCCTTGATGTACCTTGAC | 100 |
| W3 | TNF9585 | ACGTTGGATGTTCAGGCACTTGTTTGAGGG | ACGTTGGATGGGTGAGATCCTTAAGCTTCC | 98 |
| W3 | IL-10_2R420 | ACGTTGGATGAATAATTGGATCCCCTCCCC | ACGTTGGATGGAAACTGAGGCTCTTCCCAG | 98 |
| W3 | TNF9367 | ACGTTGGATGGGATGGATGGGAGAGAAAAC | ACGTTGGATGAGGAGGTTTAGCATCAGAGC | 104 |
| W3 | IL-2_12Y206 | ACGTTGGATGGAATTCTTGTGTTCACTGAG | ACGTTGGATGGTTGATACAAGTGATGATAGC | 101 |
| W3 | TNF10513 | ACGTTGGATGCTCACATCCCTGGATCTTAG | ACGTTGGATGCCCTTCAGGCTTAGAAAGAG | 116 |
| W3 | IL1a12227 | ACGTTGGATGATCCTTGTGACAGAAAGCAG | ACGTTGGATGGTAACTTACAAAGGAGCATAG | 100 |
| W3 | IL-6_10Y257 | ACGTTGGATGTTTGCAGAGGTGAGTGGTAG | ACGTTGGATGATGGCTACTGCTTTCCCTAC | 109 |
| W3 | IL-10_3M171 | ACGTTGGATGGTTCACCCCAGGAAATCAAC | ACGTTGGATGATTTTAGGATGAGCTACCTC | 119 |
| W3 | IL1aE7x255 | ACGTTGGATGGCCTTGACTCTGGAGTCTAT | ACGTTGGATGGCAAGTGACTATGAGTAAAGG | 114 |
| W3 | IL-6_8W328 | ACGTTGGATGGGTGAGAAGCTAAGGCTATG | ACGTTGGATGAATGCTAAATCCTAGCCCGC | 89 |
| W3 | 12B_03R462 | ACGTTGGATGGCAGGAACATGACTTATTGG | ACGTTGGATGTCTCGCTCAGAGCCTTTTAC | 98 |
| W4 | IL1a1619 | ACGTTGGATGTATTGGCATCTTGAGGCTGG | ACGTTGGATGCCAATCAGGAAACCTTCAAC | 102 |
| W4 | IL-10_1K362 | ACGTTGGATGCCAGTCTTCATGGAATCCTG | ACGTTGGATGCTGTGGTTGGACACTTAAGC | 107 |
| W4 | IL-6_6K372 | ACGTTGGATGTAAACCACTAAGCCACCAGG | ACGTTGGATGAAAAGCCTCTGTAGTGTGGG | 113 |

1. A method for diagnosing susceptibility to diabetes in a dog, the method comprising:
(a) (i) detecting in a sample from the dog the presence or absence of a genotype in any one of the following immune system genes: CTLA-4, IGF-2, IL-1α, IL-4, IL-6, IL-10, IL-12β, IFNγ, PTPN3, PTPN15, PTPN22, TNF, or RANTES; and/or
(ii) determining in a sample from the dog whether a genotype identified in Table 1 or 3A, or a genotype in linkage disequilibrium with said genotype identified in Table 1 or 3A, is present in an insulin or IGF gene of the dog; and/or
(iii) determining in a sample from the dog whether a genotype identified in Table 2 or 3B, or a genotype in linkage disequilibrium with said genotype identified in Table 2 or 3B, is absent in an insulin or IGF gene of the dog; and
(b) thereby diagnosing whether the dog is susceptible to diabetes.

2. The method according to claim 1, in which step (a) (i) comprises:
determining in a sample from the dog whether a genotype identified in Table 1 or 3A, or a genotype in linkage disequilibrium with said genotype identified in Table 1 or 3A, is present in the immune system gene of the dog, and/or
determining in a sample from the dog whether a genotype identified in Table 2 or 3B, or a genotype in linkage disequilibrium with said genotype identified in Table 2 or 3B, is absent in the immune system gene of the dog.

3. The method according to claim 1, in which step (a) comprises
determining in a sample from the dog whether two or more of the SNPs in the haplotypes identified in Table 3A, or a genotype in linkage disequilibrium with two or more of said SNPs, is present in the immune system gene, insulin gene and/or IGF gene of the dog, and/or
determining in a sample from the dog whether two or more of the SNPs in the haplotypes identified in Table 3B, or a genotype in linkage disequilibrium with two or more of said SNPs, is absent from the immune system gene, insulin gene and/or IGF gene of the dog.

4. The method according to claim to claim 1 wherein in step (a)
at least three different genotypes are typed, which are optionally not in linkage disequilibrium with each other, and/or
the dog is of a breed mentioned in Table 1, 2 or 4, and/or
at least one haplotype is typed that comprises at least three SNPs, and/or
in step (b) if the dog is identified as being susceptible to diabetes it is further tested to determine whether it has aberrant levels of glucose in its blood.

5. The method according to claim 1, wherein step (a) comprises contacting a polynucleotide of the dog with a specific binding agent for the genotype and determining whether the agent binds to the polynucleotide, wherein binding of the agent to the polynucleotide indicates the presence of the genotype, wherein optionally the agent is a polynucleotide which is able to bind a polynucleotide comprising the genotype but which does not bind a polynucleotide that does not comprise the genotype.

6. An isolated polynucleotide which:
comprises a genotype identified in Table 1, 2, 3A or 3B, or
is a probe or primer which is capable of detecting said genotype.

7. A kit for carrying out the method of claim 1 comprising a probe or prime capable of detecting a genotype identified in Table 1, 2, 3A or 3B.

8. A method of preparing customised food for a dog which is susceptible to diabetes, the method comprising:
(a) determining whether the dog is susceptible to diabetes by a method according to claim 1; and
(b) preparing food suitable for the dog.

9. The method according to claim 8, wherein the customised dog food comprises ingredients which prevent or alleviate diabetes and/or does not comprise ingredients which contribute to or aggravate diabetes.

10. The method according to claim 8 wherein the customised dog food comprises a suitable level of simple carbohydrate.

11. The method according to claim 8, further comprising providing the food to the dog, the dog's owner or the person responsible for feeding the dog.

12. A method of providing a customised dog food, comprising:
(a) determining whether the dog is susceptible to diabetes by a method according to claim 1; and
(b) providing food suitable for a dog that has been diagnosed as being
susceptible to diabetes by the method of claim 1 to the dog, the dog's owner or the person responsible for feeding the dog.

13. A method for identifying an agent for the treatment of diabetes in a dog, the method comprising:
(a) contacting a polynucleotide that comprises a genotype or SNP as defined in Table 1, 2, 3A or 3B with a candidate agent; and
(b) determining whether the candidate agent is capable of modulating expression from the polynucleotide.

14. (canceled)

15. A method of treating a dog for diabetes, the method comprising administering to the dog an effective amount of a therapeutic compound which prevents or treats diabetes, wherein the genome of the dog comprises a genotype or SNP as identified in Table 1 or 3A and/or does not comprise a genotype or SNP as identified in Table 2 or 3B, wherein the dog has been diagnosed as being susceptible to diabetes by the method of claim 1, and wherein the compound is optionally insulin.

16. A database comprising information relating to one or more genotypes or SNPs as identified in Table 1, 2, 3A or 3B and/or one or more genotypes which are in linkage disequilibrium with a genotype or SNP as identified in Table 1, 2, 3A or 3B and optionally also their association with diabetes.

17. A method for determining whether a dog is susceptible to diabetes, the method comprising:
(a) inputting data of one or more genotypes of the dog to a computer system;
(b) comparing the data to a computer database, which database comprises information relating to one or more genotypes or SNPs as identified in Table 1, 2, 3A or 3B and/or one or more genotypes which are in linkage disequilibrium with a genotype or SNP as identified in Table 1, 2, 3A or 3B and optionally also their association with diabetes; and
(c) determining on the basis of the comparison whether the dog is susceptible to diabetes.

18. A computer program encoded on a computer-readable medium and comprising program code which, when executed, performs all the steps of claim 17, or a computer system arranged to perform a method according to claim 17 comprising:
(a) means for receiving data of the one or more genotypes present in the dog;
(b) a module for comparing the data with a database comprising information relating to one or more genotypes or SNPs as identified in Table 1, 2, 3A or 3B and/or one or more genotypes which are in linkage disequilibrium with one or more genotypes or SNPs as identified in Table 1, 2, 3A or 3B and optionally also their association with diabetes; and
(c) means for determining on the basis of said comparison whether the dog is susceptible to diabetes.

19. (canceled)

20. (canceled)

21. (canceled)

22. A method according to claim 8, further comprising:
(a) determining whether the dog is susceptible to diabetes by a method according to claim 17 and;
(b) electronically generating a customised dog food formulation suitable for the dog;
(c) generating electronic manufacturing instructions to control the operation of food manufacturing apparatus in accordance with the customised dog food formulation; and
(d) manufacturing the customised dog food according to the electronic manufacturing instructions.

23. The computer system according to claim 18, further comprising:
(d) means for electronically generating a customised dog food formulation suitable for the dog;
(e) means for generating electronic manufacturing instructions to control the operation of food manufacturing apparatus in accordance with the customised dog food formulation; and
(f) a food product manufacturing apparatus.

24. A method of making a customised dog food formulation comprising operating a computer system according to claim 23 to thereby manufacture the customised dog food.

25. (canceled)

26. A method of selecting a dog which is not susceptible to diabetes, the method comprising determining whether the dog is susceptible to diabetes using the method of claim 1 and optionally breeding the selected dog.
